SEQUENCE LISTING (11 sequences)

SEQ ID NO: 1 - protein, 9 aa, Artificial Sequence
  CDB3 - Synthetic peptide derived from 53BP2
REDEDEIEW

SEQ ID NO: 2 - DNA, 12 bp, Artificial Sequence
  Consensus p53-binding sequence
ggaacatgttcc

SEQ ID NO: 3 - DNA, 30 bp, Artificial Sequence
  Random double stranded DNA sequence
aatatggtttgaataaagagtaaagatttg

SEQ ID NO: 4 - protein, 7 aa, Artificial Sequence
  CDB1 - Synthetic peptide derived from 53BP2
MTYSDMQ

SEQ ID NO: 5 - protein, 9 aa, Artificial Sequence
  CDB2 - Synthetic peptide derived from 53BP2
YEPQNDDEL

SEQ ID NO: 6 - protein, 20 aa, Artificial Sequence
  CDB4 - Synthetic peptide derived from p53
TPAAPAPAPSWPLSSSVPSQ

SEQ ID NO: 7 - protein, 15 aa, Artificial Sequence
  CDB5, CDB7 - Synthetic peptide derived from p53
LKSKKGQSTSRHKKL

SEQ ID NO: 8 - protein, 23 aa, Artificial Sequence
  CDB6, CDB8 - Synthetic peptide derived from p53
GSRAHSSHLKSKKGQSTSRHKKL

SEQ ID NO: 9 - protein, 14 aa, Artificial Sequence
  CDB9 - Synthetic peptide derived from p53
TPAAPAPAPSWPLS

SEQ ID NO: 10 - protein, 19 aa, Artificial Sequence
  CDB10 - Synthetic peptide derived from p53
APAAPTPAAPAPAPSWPLS

SEQ ID NO: 11 - protein, 30 aa, Artificial Sequence
  CDB11 - Synthetic fusion peptide derived from p53
PAAPAPAPSWPLSGGLKSKKGQSTSRHKKL

(19) United States
(12) Patent Application Publication
(10) Pub. No.: US 2005/0008653 A1
(43) Pub. Date: Jan. 13, 2005

(54) Stabilizing molecule

(75) Inventors: Assaf Friedler, Cambridge (GB); Alan Fersht, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 10/775,679

(22) Filed: Feb. 10, 2004

Related U.S. Application Data
(63) Continuation-in-part of application No. PCT/GB02/03668, filed on Aug. 9, 2002.

(30) Foreign Application Priority Data
Aug. 10, 2001 (GB) 0119557.7
Nov. 21, 2001 (GB) 0127917.3
May 10, 2002 (GB) 0210740.7

(51) Int. Cl. A61K 39/00

(52) U.S. Cl. 424/185.1

(57) ABSTRACT

Disclosed is a method of stabilising the native state of a polypeptide, the method comprising exposing the polypeptide to a stabilising molecule capable of binding to the polypeptide at a site which at least partially overlaps a functional site in its native state.

This application is a continuation-in-part of PCT/GB02/03668, filed Aug. 9, 2002, which claims the priority of Great Britain patent application GB 0119557.7, filed Aug. 10, 2001, Great Britain patent application GB 0127917.3, filed Nov. 21, 2001, and Great Britain patent application GB 0210740.7, filed May 10, 2002. Each of these priority documents is incorporated herein in its entirety, including figures and tables.

### FIELD

The present invention relates to polypeptides, and in particular molecules capable of stabilising native conformations of a polypeptide.

### BACKGROUND TO THE INVENTION

The maintenance of a tertiary structure is crucial for protein activity. Thus, the conformation of a plays an essential part in its ability to bind another molecule, or for its enzymatic activity. When protein conformation is disrupted, for example, by denaturation, activity may be lost.

The tumour suppressor protein p53 plays a key role in the protection of cells from cancer. It is a transcription factor, which exists in low levels in normal cells and is induced in response to DNA damage or to other conditions under which there is a danger to normal cell growth (reviewed in Hupp et al., 2000; Sigal and Rotter, 2000). Following the increase in its cellular level, p53 activates several genes, and triggers cellular processes that prevent the proliferation of the genetically impaired cells. This is achieved by mediating cell-cycle arrest or by apoptosis.

More than 50% of human cancers have mis-sense mutations in the gene coding for p53 that result in its inactivation (Hainaut and Hollstein, 2000). Nearly all such mutations are in the DNA-binding core domain (Hainaut and Hollstein, 2000). The six most frequent cancer-associated mutations are the “hot-spots” R175H, G245S, R248Q, R249S, R273H and R282W. Based on the crystal structure of p53 core domain (Cho et al., 1994), these mutations can be divided into two categories: (1) DNA-contact mutations (R248 and R273), which result in loss of DNA-binding residues, and (2) “structural mutations”, which result in structural changes in p53 core domain that can range from local distortion to complete unfolding. A new assessment of the mutation database (Bullock et al., 2000), based on thermodynamic stability and DNA binding properties of the mutants, classifies three broad phenotypes: (1) DNA-contact mutations that have little effect on folding/stability (e.g. R273H); (ii) mutations that cause a local distortion, mainly in proximity to the DNA binding site (e.g. R249S, which are usually destabilised by <2 kcal/mol); and (iii) mutations that cause global unfolding (e.g. mutations in the core domain β sandwich) that are destabilised by >3 kcal/mol (e.g. I195T).

Activation of mutant core domain by short peptides derived from the regulatory C-terminal domain of p53 (Abarzua et al., 1996; Hupp. et al., 1995; Selivanova et al., 1997; Selivanova et al., 1999) has been proposed as a means to stabilise p53. These peptides work by specifically regulating the core domain activity rather than stabilising it. Accordingly, such prior polypeptides are not relevant to the invention disclosed here.

It is a problem in the art to provide a means to rescue p53 mutants, and other mutants in tumour suppressor proteins, to restore tumour suppression activity for cancer therapy. Mutations in oncogenes are also known to cause tumour activity. It is a further problem in the art to provide means to rescue such oncogenic mutations.

### SUMMARY

The present inventors have realised for the first time that different classes of mutants of tumour suppressor proteins and oncogene proteins require different rescue strategies. In order to rescue DNA contact mutants of tumour suppressor proteins, for example, there is a need to introduce functional groups that will establish new contacts with the DNA, compensating for the missing contacts. We have discovered that rescue of globally unfolded or locally distorted mutants may be achieved by stabilisation that will lead to refolding of the mutant, which in turn will lead to restoration of the wild-type p53 activity.

It has been reported that the rescue of mutant p53 may be achieved by small molecules, e.g. CP-31398. CP-31398 is said to stabilise only newly synthesised p53 that is in the active conformation, which then allows the time dependent accumulation of this fraction (Foster et al., Science, vol 286, 1999, 2507-2510). However, we and others have not found that CP-3 1398 does not in fact work to stabilise active conformations of p53.

We therefore provide for the first time a molecule which is capable of binding a native conformation of a protein, such that the binding stabilises the native conformation. We term such a molecule a “stabilising molecule”. Stabilisation of the native conformation enables the equilibrium between an unfolded, denatured and/or inactive conformation of the polypeptide and a properly folded, native and active form to shift towards the latter. Accumulation of native protein therefore results. The stabilising molecules according to the invention advantageously do not bind the denatured/inactive form of the peptide, thus preferentially stabilising the active conformation.

Thus, in a first aspect, the present invention provides a method of stabilising the native state of a polypeptide, the method comprising exposing the polypeptide to a stabilising molecule capable of binding to the polypeptide at a site which at least partially overlaps a functional site in its native state.

According to the above aspect of the invention, advantageously the polypeptide is reversibly denatured such that it exists in a native state, in which the stabilising molecule does not bind to the polypeptide in its denatured state.

There is provided, according to a further aspect of the present invention, a method of increasing the concentration of a native state of a reversibly denatured polypeptide in a system, in which the system comprises the polypeptide in a first native state and a second denatured state, the method comprising: (a) providing a stabilising molecule which binds to the polypeptide at a site which at least partially overlaps a functional site in the first native state and thereby stabilising the first native state of the polypeptide; and (b) allowing the stabilising molecule to bind to the polypeptide.

According to a further aspect of the present invention there is provided a method of restoring a wild-type phenotype of an organism comprising a mutation in a polypeptide, in which the mutation leads to denaturation of the polypeptide and a mutant phenotype, the method comprising exposing the organism or part of the organism to a stabilising molecule which binds to the polypeptide at a site which at least partially overlaps a functional site in its native state and thereby stabilises the native state of the polypeptide.

As a further aspect of the present invention, there is provided a method of treatment of a disease in a patient, in which the disease is caused by or associated with a mutation in a polypeptide which leads to denaturation of the polypeptide, the method comprising administering to the patient a stabilising molecule which binds to the polypeptide at a site which at least partially overlaps a functional site in its native state and thereby stabilises the native state of the polypeptide.

In a preferred embodiment, the stabilising molecule is not a natural binding partner of the polypeptide. Preferably, the stabilising molecule consists of a fragment of a natural binding partner of the polypeptide. More preferably, the stabilising molecule is a polypeptide engineered to include a polypeptide binding domain, preferably a binding loop, of a natural binding partner of the polypeptide.

As used herein, the phrase “natural binding partner” refers to a molecule that specifically binds to the native state of a given polypeptide in vivo in a wild-type cell or organism.

As used herein, the term “specifically binds” refers to the interaction of two molecules, e.g., a ligand and a protein or peptide, wherein the interaction is dependent upon the presence of particular structures on the respective molecules. For example, when the two molecules are protein molecules, a structure on the first molecule recognizes and binds to a structure on the second molecule, rather than to proteins in general. “Specific binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

As used herein, the term “fragment of a natural binding partner” refers to a portion of a natural binding partner which retains the ability to bind a functional site on a target polypeptide. Methods for determining the binding of a “fragment of a natural binding partner” to a target polypeptide are known in the art and described herein.

As used herein, the phrase “polypeptide binding domain of a natural binding partner polypeptide” refers to the specific structure of a natural binding partner for a target polypeptide that physically interacts with that target polypeptide. The polypeptide binding domain retains the ability to specifically bind the target polypeptide in isolation from the remainder of sequences of the natural binding partner. Binding domains frequently comprise polypeptide loops or series of loops which protrude from the surface of the natural binding partner and provide contact surfaces with a target polypeptide. Thus, where, for example, the binding domain for a natural binding partner consists of a polypeptide loop, helix or other polypeptide structure, that polypeptide loop, helix or other structure, that, in isolation from the remainder of the natural binding partner polypeptide can bind the target polypeptide, is a “polypeptide binding domain of a natural binding partner polypeptide.”

The stabilising molecule may be exposed to polypeptide or the system in presence of a natural binding partner of the polypeptide. Preferably, the affinity of binding between stabilising molecule and the polypeptide or binding site is less than the affinity of a natural binding partner of the polypeptide and the polypeptide or the binding site. More preferably, binding between the stabilising molecule and the binding site stabilises the polypeptide to enable binding between the polypeptide and a natural binding partner. Most preferably, binding between the polypeptide and the natural binding partner stabilises the native state of the polypeptide.

As used herein, the phrase “is less than,” when used in reference to binding affinity for a target polypeptide, means that for equal concentrations of a stabilizing molecule and a natural binding partner, at equilibrium, there is more (e.g., at least 1% more) of the natural binding partner than the stabilizing molecule bound to the target polypeptide.

There is provided, according to yet a further aspect of the present invention, a method of assisting the binding between a polypeptide and a natural binding partner for the polypeptide, the method comprising stabilising a native state of the polypeptide by a method as herein described, and exposing the stabilised polypeptide to the natural binding partner.

The present invention, in yet a further aspect, provides a method of assisting the binding between a polypeptide and a first molecule, in which the polypeptide exists in a native state and a denatured state, the method comprising: (a) providing a second stabilising molecule capable of binding to a site which at least partially overlaps a functional site in the native state of the polypeptide; (b) allowing the second stabilising molecule to bind to the polypeptide to form a complex and thereby stabilising the native state of the polypeptide; (c) exposing the polypeptide and bound second stabilising molecule complex to the first molecule; and (d) allowing the first molecule to bind to the polypeptide and thereby displacing the second stabilising molecule.

As used herein, the term “assisting the binding” means that the physical interaction between a polypeptide and a natural binding partner occurs more readily in the presence of a stabilizing molecule than in its absence. The assistance in binding occurs more readily if the equilibrium binding concentration of target polypeptide and the natural binding partner is higher (e.g., at least 10% higher) in the presence, as opposed to the absence of the stabilizing molecule.

The functional site preferably comprises or at least partially overlaps with o a structural domain, a protein binding domain, a nucleic acid binding domain, or an active site of an enzyme. More preferably, the functional site is essential to the structure or activity, or both, of the polypeptide.

In a highly preferred embodiment of the invention, the polypeptide comprises an oncogenic protein or a tumour suppressor protein. Preferably, the polypeptide is p53. More preferably, the polypeptide is p53 which comprises a mutation, preferably R175H, G245S, R248Q, R249S, R273H, R282W and I195T in which the mutation leads to reversible denaturation of the polypeptide.

The stabilising molecule may comprise a CDB3 polypeptide having the sequence REDEDEIEW. Advantageously, the peptide may be labelled with fluorescein at its N terminus. Such a peptide is referred to as FL-CDB3 and has the sequence F1-REDEDEIEW.

In a further aspect of the present invention, there is provided a stabilising molecule which binds to and stabilises the native state of a polypeptide, but not a denatured state of the polypeptide, in which the stabilising molecule binds to a site which at least partially overlaps a functional site of the polypeptide, and in which the stabilising molecule does not consist of a natural binding partner of the polypeptide.

Preferably, the polypeptide is p53. More preferably, the polypeptide is p53 which comprises a mutation, preferably R175H, G245S, R248Q, R249S, R273H, R282W and I195T in which the mutation leads to reversible denaturation of the polypeptide. Most preferably, the stabilising molecule comprises a CDB3 polypeptide having the sequence REDEDEIEW.

According to a further aspect still of the present invention, we provide a method of identifying a stabilising molecule capable of stabilising a polypeptide, in which the polypeptide may be reversibly denatured such that it exists in a native state and a denatured state, the method comprising the steps of: (a) providing a native state of the polypeptide comprising a functional site; (b) exposing the polypeptide to a candidate stabilising molecule; (c) selecting a candidate stabilising molecule which binds to a site which at least partially overlaps a functional site of the native state of the polypeptide; and (d) determining whether such binding stabilises the native state of the polypeptide.

There is provided, according to a further aspect of the invention, a method of identifying a stabilising molecule capable of stabilising a polypeptide, in which the polypeptide may be reversibly denatured such that it exists in a native state and a denatured state, the method comprising the steps of: (a) identifying a functional site of the polypeptide and providing a polypeptide fragment comprising the functional site; (b) selecting a candidate stabilising molecule which binds to the polypeptide fragment at a site which at least partially overlaps a functional site; (c) determining whether the selected candidate stabilising molecule stabilises a native state of a polypeptide.

The polypeptide fragment may comprise the functional site includes a binding site for a natural binding partner of the polypeptide.

There is provided, in accordance with yet a further aspect of the present invention, a stabilising molecule capable of stabilising a polypeptide, which is identified by a method according to the previous two aspects of the invention.

A stabilising molecule as described here preferably comprises a natural or derivatised carbohydrate, protein, polypeptide, peptide, glycoprotein, nucleic acid, DNA, RNA, oligonucleotide, protein-nucleic acid (PNA) or a small molecule compound. The methods as described here may employ such a derivatised or natural stabilising molecule. More preferably, the stabilising molecule is derivatised with a sugar, phosphate, amine, amide, sulphate, sulphide, biotin, a fluorophore or a chromophore. Most preferably, the stabilising molecule is derivatised using a fluorophore, preferably fluorescein.

As used herein, the term “derivatised” refers to the covalent linkage of a chemical moiety to a given molecule. Derivatisation can confer desired properties upon a stabilising molecule as described herein. Desired properties include, for example modified solubility or localization, detectability, ability to be attached to a substrate or another molecule, and modified stability, among others. For example, if desired, a stabilizing molecule as described herein can be derivatised with a sugar, phosphate, amine, lipid, amide, sulphate, sulphide, biotin, a fluorophore and a chromophore, among others.

The binding of a stabilising molecule to the polypeptide may be detected using NMR spectroscopy, preferably heteronuclear NMR spectroscopy, fluorescence anisotropy, surface plasmon resonance, or Differential Scanning Calorimetry (DSC).

As a further aspect of the invention, we provide a stabilising molecule according to the relevant previous aspects of the invention for use in the treatment of a disease.

The present inventors have found that a stabilising molecule according to the present invention may be of particular use in inducing the onset or progression of apoptosis.

Thus, in a further aspect, the present invention provides a method for inducing the onset or progression of apoptosis in one or more cells comprising the steps of treating those one or more cells with a stabilising molecule according to the present invention.

In a further aspect still, the present invention provides the use of a stabilising molecule in the preparation of a medicament for inducing the onset or progression of apoptosis in one or more cells comprising the steps of treating those one or more cells with a stabilising molecule.

According to the above aspects of the invention, preferably the stabilising molecule is a CDB3 peptide as herein described.

As used herein “apoptosis” or cell death refers to a controlled intracellular process characterised by the condensation and subsequent fragmentation of the cell nucleus during which the plasma membrane remains intact. A cascade of enzymes including caspases that cleave at aspartic acid residues is activated in the process.

Methods for inducing apoptosis are familiar to those skilled in the art, and include without limitation, exposure to chemotherapy or radiotherapy agents and withdrawal of obligate survival factors, if applicable. Differences between treated and untreated cells indicates effects attributable to the test compound.

Methods for measuring apoptosis are familiar to those skilled in the art and are described herein.

According to a further aspect of the invention, there is provided a pharmaceutical composition comprising a stabilising molecule as described herein together with a pharmaceutically acceptable carrier, diluent or exipient.

According to a further aspect of the present invention, we provide the use of stabilising molecule as described herein in the manufacture of a medicament for treatment of a disease.

According to a yet a further aspect, the present invention provides the use of a stabilising molecule as described herein in the treatment of disease.

Advantageously, the disease is cancer.

### BRIEF DESCRIPTION OF THE FIGURES

FIG. 1 shows the crystal structure of the p53 core domain (blue)-53BP2 (red) complex (coordinates taken from Gorina and Pavletich, 1996) with the three 53BP2 derived peptides synthesized for this study highlighted: CDB1 (residues 422-428)—green, CDB2 (residues 469-477)—yellow, CDB3 (residues 490-498)—purple. Picture is generated using swissPDB viewer (Guex and Peitsch, 1997).

FIG. 2 shows a 1H, 15N HSQC spectra of p53 core domain in the presence (red) and the absence (black) of CDB3. Selected residues that show significant chemical shift deviation in presence of CDB3 are highlighted.

FIG. 3 shows the binding of p53 core domain to immobilised peptides analysed by surface plasmon resonance. (a) Screening for p53 core domain binding peptides. Biotinylated peptides are immobilised on a streptavidin BIAcore chip and p53 core domain (7.21 μM) is injected. The values shown are normalised by the response upon p53 injection to the flow channel without any immobilised peptide. (b) Concentration dependence of p53 core domain binding to immobilised CDB3. (c) Titration of CDB3 binding to p53 core domain by competition BIAcore. The concentration of free p53 core domain (reflected by association rate in binding to immobilized CDB3) is analyzed by BIAcore after incubation of 0.2 μM p53 core domain and various concentrations of free CDB3.

FIG. 4 shows the chemical shift changes (∂) in p53 core domain upon binding to CDB3. (a) ¹H and ¹⁵N Chemical shift deviations plotted against residue number. Deviations above 5 times the standard deviation (∂>0.25 ppm for ¹⁵N and ∂>0.05 ppm for ¹H) are considered significant (white background). ∂ differences between 2.5 times and 5 times the standard deviation (0.125<∂<0.25 ppm for ¹⁵N, 0.025<∂<0.05 ppm for ¹H) are considered as minor (light grey background), and ∂ differences below 2.5 times the standard deviation (<0.125 ppm for ¹⁵N and ∂<0.025 ppm for ¹H) are considered insignificant (dark grey background). (b) Chemical shift changes in the p53 core domain structure upon CDB3 binding. Residues with significant chemical shift changes are coloured blue, residues with minor changes are coloured purple and residues with no change are coloured yellow. CDB3 in its original position in the 53BP2-p53 complex is shown in red (coordinates taken from (Gorina and Pavletich, 1996)).

FIG. 5 shows the CDB3 binding to p53 core domain analysed by anisotropy and fluorescence. (a) Wild-type and mutant p53 core domain are titrated into a fluorescein-labeled CDB3 (4.6 μM). Changes in anisotropy are monitored and analysed. (b) Competition experiment where unlabeled or biotinylated CDB3 are titrated into 0.50 μM fluorescein-labeled CDB3 and 2.0 μM p53 core domain wild-type (▪ and □, 0.2 mM and 2.6 mM unlabeled CDB3, respectively, and ●, 0.24 mM biotinylated CDB3).

FIG. 6 shows the stabilisation of p53 core domain by FL-CDB3. (a) differential scanning calorimetry. The apparent Tₘ of wild-type and R249S core domain in the presence or absence of FL-CDB3 is determined as described in materials and methods. For the wild-type core domain Tₘ=40.1° C. in the absence of the peptide and 41.6° C. in its presence. For R249S Tₘ=34.9° C. in the absence of the peptide and 35.9° C. in its presence. Raw data are shown and are offset for clarity. (b-c) Urea dependence of p53-CDB3 binding. Wild-type p53 core domain is titrated into fluorescein-labeled CDB3 in presence of increasing urea concentrations, and changes in anisotropy are monitored. (b) anisotropy titration curves under various urea concentrations (c) log K_{d} for the p53 core domain CDB3 interaction versus urea concentration (d) CDB3 induces refolding of p53 core domain. Wild-type p53 core domain is pre-incubated overnight with 3 M urea, then mixed with fluorescein-labeled CDB3 and the anisotropy change over time is monitored. As a control, the same protein is mixed with 3M urea and with fluorescein-labeled CDB3 without pre-incubation and anisotropy changes over time are monitored.

FIG. 7 shows the “Chaperone” strategy for rescue of p53. (a) DNA competes with FL-CDB3 on p53 core domain binding. 30-mer Gadd45 DNA (+25 μM, =5 μM) was titrated into a mixture of p53 core domain-FL-CDB3 as described in materials and methods. (b) CDB3 restores DNA binding to the I195T mutant. I195T (10 μM) was preincubated for 1 h in the presence (▪) and the absence (x) of 100 μM CDB3 and titrated into 15 nM fluorescein-labeled 30-mer Gadd45 DNA. Dissociation constants were calculated from a fit to a 1:1 binding model. (c) A schematic model of the proposed mechanism of action for CDB3. See text for details.

FIG. 8. Distribution of FL-CDB3 in cells after treatment with peptide for 24 h. The nuclei are visible in blue (staining with Hoechst), the peptide is green. Top left: H1299 cells containing p53 R175H. FL-CDB3 was localised in nuclei and large deposits could be seen in a nucleolus. Top right: cytoplasmic distribution was also observed in some cases. Middle: after combined delivery with Lipofectamine 2000™, the peptide was located in the cytoplasm, although some nuclear fraction was present as well. Bottom left and right: distribution of the peptide in parental p53-null H1299 cells. It appears that in p53 null cells peptide is localised mostly in cytoplasm (H1299), although in some cells nucleolar localisation is also evident (H12991-1). The peptide remained visible for at least 48 h.

FIG. 9. Detection of induced protein expression by Western blots after 24 h incubation with FL-CDB3. Frames A, C, and D: Treatment with FL-CDB3 restored the ability of p53 mutants His175 and His273 to activate the transcription of endogenous genes p21 and Mdm-2. Lung carcinoma cells H1299 transfected with His175 p53 mutant and parental nontransfected cells were treated with the amounts of peptide indicated below, incubated for 24 h and tested for p53, p21, and Mdm-2 protein expression. The levels of actin show the equal loading of protein. Notably, mutant p53 levels were remarkably increased. B: Treatment with FL-CDB3 induces wtp53 in colon carcinoma HCT 116 cells and activates expression of Mdm-2 and p21. No induction of p21 nor Mdm-2 was observed in the absence of p53 expression in HCTp53−/− cells. For A and B: Lane 1 was the control with no FL-CDB3; Lane 2 was 24 h post treatment with 10 μg/mL FL-CDB3. For C and D, Lane 1 was the control (no FL-CDB3); Lane 2, 10 μg/mL FL-CDB3; and Lane 3, 1 μg/mL FL-CDB3. The treatment with peptide was performed either with or without Lipofectamine. All the data presented here were obtained after treatment without Lipofectamine, except frames C and D. The induction of p53 target genes in C and D is seen to be dependent on the concentration of FL-CDB3.

FIG. 10. FACS analysis of effects of FL-CDB3 on cell cycle. We treated tumour cells with 10 μg/mL of peptide and analysed the cell cycle distribution and cell death (as subG1 fraction) 24 h post treatment using FACS analysis. The left hand side of each pair of panels is the control without FL-CDB3. In one experiment, the percentage of dead cells was determined by trypan blue exclusion: the number of dead cells in H1299-His175 cells before treatment was 5%, after treatment, 37%; in control H1299 (p53^{˜}), before 3%, after treatment 11%; in Saos-2-His273 cells, before 3%, after 28%; in control Saos-2(p53^{˜}); before treatment 3%, after 13%.

### DETAILED DESCRIPTION OF THE INVENTION

The invention relies on the provision of a stabilising molecule which is capable of binding to a native form of a polypeptide, thereby stabilising it.

Where the polypeptide exists in equilibrium between a native, properly folded or active form and a denatured, unfolded or inactive form, binding of the stabilising molecule to the native form of the polypeptide stabilises it and drives the equilibrium towards the folded, active or native form. Thus, the stabilising molecule is capable of increasing the relative concentration of a native form of a polypeptide as compared to a denatured form. Such a stabilising molecule will bind the native, but not the denatured state of the polypeptide. The law of mass dictates that in such a case the equilibrium will be shifted towards the native state and the amount of active protein will increase.

Preferably, the polypeptide is reversibly denatured. In other words, a proportion of the polypeptide molecules in any system is in the native, folded, or active form, and a proportion of the polypeptide molecules is in the inactive, unfolded (whether partially or fully) or denatured form. Such denaturation may arise though various means, and the invention is suitable for use in any of these situations. Thus, the polypeptide may be exposed to an environment which results in its denaturation; for example, by being exposed to a non-physiological environment. The polypeptide may be oxidised by exposure to air, or denatured by exposure to heat, high or low salt concentrations, etc. The polypeptide may be denatured by virtue of a co-factor being removed from it.

In a highly preferred embodiment, however, the reversible denaturation of the polypeptide results from genetic mutation. Thus, a mutation in the sequence of the polypeptide results in its destabilisation and tendency to denature. Preferably, such a mutation results in loss of activity of the polypeptide. The mutation may result in a mutant phenotype of the polypeptide, or cell, tissue or organism comprising the mutant phenotype, such a mutant phenotype being different in some detectable way from a wild-type phenotype associated with a unmutated or wild-type polypeptide. The methods of our invention are therefore suitable for rescuing such a mutant phenotype. These methods may also be used to rescue a mutant form of a protein, for example, an oncogene protein or a tumour suppressor protein, by a stabilising molecule binding to the native state of the protein, but not the denatured state, and thereby shifting the equilibrium that exists between the two forms to the native state.

It is known that mutated forms of oncogenes and tumour suppressor proteins are involved in tumourogenesis. As noted above, such mutations may lead to partial denaturation of the polypeptide and loss of activity. Therefore the methods of our invention are suitable for stabilising such mutated oncogenes and/or tumour suppressor proteins and restoring wild-type activity. Accordingly, the methods described here are suitable for rescuing wild-type activity of oncogenes and tumour suppressors, and hence of preventing tumourogenesis and/or cancer. Preferably, the oncogene comprises p21ras, or any other oncogene known in the art. Preferably, the tumour suppressor comprises p53 or retinoblastoma protein. The p53 may comprise a mutation leading to partial denaturation, preferably reversible denaturation. Examples of such mutations include R175H, G245S, R248Q, R249S, R273H and R282W.

Furthermore, it is known that many diseases are caused by or associated with polypeptide mutations, which mutations may lead to destabilisation and reversible denaturation of the protein. Administration of a stabilising molecule as described here to a patient suffering from such a disease will stabilise the native form of the polypeptide, and increase the amount or relative concentration of the native form over the denatured form. Accordingly, administration of stabilising molecules may be used to treat diseases associated with or caused by such mutations.

In a highly preferred embodiment, the stabilising molecule binds to a site which comprises or at least partially overlaps a functional site in the polypeptide. Preferably, the site at which the stabilising molecule binds overlaps or consists of the functional site. Such a functional site preferably comprises a site which is essential for a relevant activity of the polypeptide. The functional site may also be essential for the structure of the polypeptide. The functional site may be an interaction site, which interacts with another molecule in the cell, such as a natural binding partner of the polypeptide including another polypeptide, a small molecule, a ligand, a macromolecule, a nucleic acid, etc.

Examples of such functional sites include active sites, or substrate binding sites, where the polypeptide is an enzyme. In the case of binding proteins, the functional site comprises, or at least overlaps, a binding site or binding domain of the polypeptide. Thus, in the case of nucleic acid binding sites, the functional site comprises a nucleic acid binding site, such as a DNA binding site in a DNA binding protein, or an RNA binding site in a RNA binding protein. Where the polypeptide interacts with another polypeptide, i.e., has polypeptide binding activity, the functional site preferably comprises a polypeptide interaction domain or sequence, i.e., it includes, overlaps, or is a sequence which interacts with another polypeptide.

Preferably, therefore the stabilisation of the native state of the polypeptide enables the binding of another molecule to the polypeptide. This other molecule is preferably a different molecule or unrelated molecule to the stabilising molecule. Thus, stabilisation of the polypeptide by the stabilising molecule preferably enables a proper conformation of the functional site to be maintained in the polypeptide, to allow the binding of the other molecule. Preferably, the other molecule is a natural binding partner of the polypeptide, for example, a DNA where the polypeptide is a DNA binding protein.

Thus, the stabilising molecule is capable of competing with the binding of a natural binding partner of the polypeptide for binding to the polypeptide or the functional site. Preferably, however, the affinity of binding of the stabilising molecule to the polypeptide is less than the affinity of binding of a natural binding partner to the polypeptide. Thus, the natural binding partner is capable of displacing the stabilising molecule from the functional site, or the binding site of the natural binding partner. Thus, in this preferred embodiment, the binding of the stabilising molecule to the polypeptide stabilises the native state of the polypeptide for long enough to enable binding of the natural binding partner to the polypeptide.

Binding of the stabilising molecule to the native state shifts the equilibrium to this state. Preferably, therefore, the stabilising molecule does not require energy for its stabilising activity. The stabilising molecule as described here does not actively refold the polypeptide, in contrast to classic chaperone activity.

Preferably, the functional site exists only in the native, active or properly folded form of the polypeptide. More preferably, the functional site does not exist in the denatured form of the polypeptide. Preferably, the affinity of binding of the stabilising molecule to the native form of the polypeptide is greater than the affinity of binding to the denatured form of the polypeptide. In a highly preferred embodiment, the stabilising molecule substantially only binds to the native form and not the denatured form of the polypeptide.

While small stabilising molecules are included, preferred stabilising molecules comprise polypeptides, preferably derived from natural binding partners of the polypeptide to be stabilised. This overcomes the difficulty and expense of synthesising small molecules. In addition, it is often difficult to scale up the synthesis procedure of identified small molecules.

Although natural binding partners of the polypeptide to be stabilised may be used as stabilising molecules, a highly preferred embodiment relies on the use of a stabilising molecule which is not a natural binding partner of the polypeptide. By this we mean that the stabilising molecule is preferably an engineered molecule, which does not exist in nature, but which is capable of binding to the polypeptide in its native form and stabilising it. Engineered stabilising molecules may be generated by means known in the art, including recombinant DNA technology. They preferably comprise or consist of fragments of natural binding partners, preferably fragments comprising binding activity. Thus, for example, where the stabilising molecule is a polypeptide, this suitably consists of or comprises a polypeptide binding sequence, loop or domain. An example of this is a stabilising molecule consisting of CDB3, which is a fragment of a p53 binding polypeptide 53BP2 (accession number NM_{—}005426.1).

One skilled in the art will appreciate that the stabilising molecule may act in isolation in the rescue of mutant proteins. Alternatively, it may act in conjunction with another peptide, or other stabilising molecule in the rescue of the protein. There may be an additive effect between one or more peptides or molecules, alternatively they may act synergistically.

In a preferred embodiment, the polypeptide is an oncogenic protein or a tumour suppressor protein, preferably a mutant oncogenic protein or a mutant tumour suppressor protein. Advantageously, the protein is p53, preferably a mutant of p53. The tumour suppressor protein may comprise retinoblastoma protein (RB-). Those skilled in the art will appreciate this list is by no means exhaustive.

The binding of the stabilising molecule to the native polypeptide may detected using any suitable means known in the art. Preferred means include physical methods such as NMR spectroscopy. In a preferred embodiment the NMR involves the use of heteronuclear NMR spectroscopy. The binding may also be detected using surface plasmon resonance. Alternatively, the binding of the stabilising molecule to the native form of the polypeptide is detected using Differential Scanning Calorimetry (DSC) and/or fluorescence anisotropy. All of these methods will be familiar to those skilled in the art and are described in detail in this document.

In an alternative embodiment, the binding of the stabilising molecule to each state of the polypeptide, i.e., native or denatured, may be detected by examining the fraction of the polypeptide sample which expresses an epitope for one or more monoclonal antibodies, which epitopes are only present in one form of the polypeptide. Other suitable methods for detecting conformational changes in proteins include, but are not limited to electrophoresis and thin-layer chromatography. Those skilled in the art will be aware of other suitable methods.

In a particular embodiment, the polypeptide comprises a DNA binding protein. A mutated form of the DNA binding polypeptide comprises a denatured form which is incapable of binding DNA. A stabilising molecule is provided which binds an unfolded or distorted oncogenic protein which is unable to bind DNA, and shifts the equilibrium that exists between the denatured state and the native “wild-type” state towards the latter. DNA can then bind the mutated protein, displacing the molecule, which is preferably a peptide, so that it is free again to bind another protein molecule.

In a preferred embodiment, denaturation of a polypeptide arises from mutation in the polypeptide. Such mutations may cause a local structural distortion, compared with the wild type. In the context of DNA binding proteins, mutant proteins may comprise mutations mainly in close proximity to the DNA binding site. Typically mutant proteins of this type will be destabilised by less than 2 kcal/mol. The term “mutant protein” also includes within its scope proteins possessing those mutations which cause global unfolding, for example in the core domain beta sandwich of a DNA binding protein such as p53. Typically mutant proteins of this type will be destabilised by greater than 3 kcal/mol. The term “mutant protein” as herein defined does not include within its scope contact mutants which have little effect on folding or stability of the protein.

Core domain in the context of this document describes a region of a protein, preferably a p53 protein, which generally has a defined secondary and/or tertiary amino-acid conformation. It is generally structurally stable in the absence of the remainder of the protein, and advantageously confers structural stability on the protein. Mutations within this region will often cause structural instability and partial or total unfolding of the protein and/or loss of functional activity.

An oncogenic protein includes a protein which plays a role in the onset or maintenance of cancer. In addition, in the context of this document the term “oncogenic protein” also includes within its scope proteins which have a role in the suppression and/or prevention of the onset or maintenance of cancer. Oncogenic proteins of this sort include tumour suppressor proteins, such as p53.

A polypeptide in a “native state” may include a conformation which corresponds to the conformation of a wild-type polypeptide. The polypeptide may comprise a well-defined three dimensional structure, and may comprise a native biological and/or binding activity. A “denatured polypeptide” in the context of this document describes a protein which is at least partially structurally distorted, and/or unfolded as compared with the native/wild-type protein. Generally, denatured proteins have at least a partial loss and/or altered biological activity as compared with the wild-type or native protein.

As used herein in the context of binding of a stabilizing molecule at a site which at least partially overlaps a functional site, the term “overlaps” means that the stabilizing molecule physically contacts at least one amino acid within a functional site of a target polypeptide. Alternatively, or in addition, the binding in this context “overlaps” a functional site when the stabilizing molecule competes with a natural binding partner for binding to a given functional site. The phrase “at least partially” in the context of overlap emphasizes that the stabilizing molecule need not bind or occupy the entire functional site, nor contact all of the amino acids contacted by a natural binding partner, but rather, must contact at least one amino acid in the functional site and/or must compete with a natural binding partner for binding to the given functional site.

A polypeptide, preferably a mutant polypeptide, is “rescued” when the proportion of native (versus denatured) polypeptide under a certain set of conditions is increased as compared to an un-rescued polypeptide. The normal biological and/or binding activity and/or structure native form of the protein may be restored, preferably to a substantial number of polypeptide molecules. Advantageously, a proportion of polypeptide molecules which are rescued have the same structural conformation as the wild-type or native protein. Preferably, the methods described here are capable of increasing the proportion of native polypeptide by 10%, 20%, 30%, 40%, 50%, 60%, 70%, 80%, 90% or more. Preferably, 50% or more, preferably 60%, 70%, 80%, 90%, 95% or more of the molecules in a polypeptide population are in the native state. Most preferably, substantially all of the polypeptide molecules in a population are in the native state.

As used herein, the phrase “increasing the concentration” when used in reference to the native state of a polypeptide means that the equilibrium between the native state and the denatured state of a reversibly denatured polypeptide is shifted, in the presence of a known or candidate stabilizing molecule, such that the proportion of the polypeptide in the native state is higher (i.e., at least 10% higher, and preferably more, e.g., 10%, 20%, 30%, 40%, 50%, 60%, 70%, 80%, 90% or more) in the presence of the stabilizing molecule than in its absence.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology, 4^{th} ed. (1999) John Wiley & Sons, Inc. which are incorporated herein by reference) and chemical methods. In addition, Harlow and Lane, A Laboratory Manual, Cold Spring Harbor, N.Y., is referred to for standard Immunological Techniques.

### Stabilising Molecule

Stabilising molecules are capable of binding to the native form of the polypeptide in question. The binding site of the stabilising molecule may overlap at least partially with a functional site of the protein, or it may comprise or be comprised in the functional site.

The binding of the stabilising molecule to the polypeptide must be such that it stabilises the native form of the polypeptide. Thus, the binding site for the stabilising molecule needs to be present in the native form of the polypeptide. Preferably, the binding site of the stabilising molecule is not present in a denatured form of the polypeptide. However, where this is the case, the stabilising molecule should bind to the native form with a higher affinity than the denatured form; i.e., it should bind preferentially to the native form of the polypeptide.

The binding of the stabilising molecule to the binding site, or the polypeptide, may occur by any known mechanism, e.g., by ionic, covalent, polar bonds, salt bridges, van der Waals interactions, hydrophobic interactions, etc. The stabilising molecule may stabilise the polypeptide by maintaining it in a certain conformation, or by inducing a conformational change, etc. The mechanism by which the stabilising molecule stabilises the native form of the polypeptide is not crucial, only that it does so when bound to the polypeptide. Preferably, the stabilising molecule does not bind to the denatured form, or where it does so (with less affinity as noted above), it does not stabilise the denatured form to any substantial extent. Where stabilisation does occur, the denatured form is stabilised to a lesser extent than stabilisation of the native form.

Where reference is made to “stabilisation” of a polypeptide or a form of a polypeptide, this is to be taken to mean that the polypeptide or form is less susceptible to unfolding or conversion into another form than otherwise. A stabilised polypeptide will preferably have a higher melting point (Tₘ) than an unstabilised polypeptide. Thus, stabilisation of a polypeptide raises its apparent Tₘ. Preferably, the Tₘ is raised by 0.5, 1, 1.5, 2, 2.5, 3, 4, 5, 6, 7, 8, 9, 10, 15, 20, 25, 30 or more degrees. Means for making Tₘ measurements are known in the art.

Stabilisation may also be assessed in terms of kCal/mol or equivalent measurements, for example kJ/mol. Preferably, a stabilised polypeptide has an increase of 0.5, 1, 1.5, 2, 2.5, 3 or more kCal/mol or kJ/mol compared to an unstabilised molecule.

Stabilisation may also be used to refer to a shift in equilibrium from one form of the polypeptide to another. Thus, stabilisation of a form of a polypeptide may result in a higher proportion of polypeptides in a relevant population being in that form.

Furthermore, stabilisation may also be assessed by the amount of time a particular form of polypeptide exists in one form compared to another.

Stabilising molecules may be identified by various means. Suitable candidates may be identified from those molecules which bind to a polypeptide close to, or at an active or functional site. Candidates may be identified from known molecules which bind to the polypeptide in question. Such molecules may comprise polypeptides, small molecules, nucleic acids, etc. Fragments of such molecules, for example, fragments of a known binding polypeptide comprising for example the binding site, may be generated and screened. Fragments of the polypeptide to be stabilised itself may be generated as candidates also. These can suitably include fragments within the polypeptide which interact with the functional site to stabilise it, or which are involved in stabilising the polypeptide as a whole, preferably by binding close to or at the binding site. In the specific Examples presented below, candidate binding peptides are generated from the p53 molecule itself, and assayed for stabilisation of p53.

Assays to identify such molecules may be used, as known in the art. For example, a library (such as a combinatorial library, or a nucleic acid library, or a polypeptide library, which may be expressed on a host by for example phage display) may be screened for binding to the polypeptide or a fragment of the polypeptide comprising the functional site. Mass screening may involve the use of arrays of candidate molecules, or polypeptides, or fragments of these. Database searches for known binding molecules may be carried out to identify candidates. Binding assays may be carried out using ELISA, gel shift assays, or other methods as set out in greater detail below.

A “functional site” as the term is used here, refers to a site which is involved in maintaining a relevant activity of the polypeptide. Functional sites for many polypeptides are known, and are listed in protein databases or in the literature for the relevant polypeptide. Such functional sites may include binding sites, for example, sites which modulate binding of the polypeptide to another molecule, such as another polypeptide, nucleic acid, or other molecule such as a ligand. The functional site may also include a site essential for the structure or activity of the polypeptide, whether this is binding activity, enzymatic activity or any other kind of activity. Methods for assaying such activities will depend on the particular activity concerned, and will be known in the art.

Candidate molecules which are identified may be tested for their ability to stabilise the native form of a polypeptide, by, for example, comparing the melting point of a polypeptide compared to a complex of the polypeptide and the candidate molecule.

### Nature of Stabilising Molecule

As used herein, the term “stabilising molecule” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. A stabilising molecule may be in solution or in suspension (e.g., in crystalline, colloidal or other particulate form). The stabilising molecule may be in the form of a monomer, dimer, oligomer, etc, or otherwise in a complex.

The stabilising molecule may be labelled by a radio-isotope as known in the art, for example ³²P or ³⁵S or ⁹⁹Tc, or a molecule such as a nucleic acid, polypeptide, or other molecule as explained below conjugated with such a radio-isotope. The stabilising molecule may be opaque to radiation, such as X-ray radiation. The stabilising molecule may also comprise a targeting means by which it is directed to a particular cell, tissue, organ or other compartment within the body of an animal. For example, the stabilising molecule may comprise a radiolabelled antibody specific for defined molecules, tissues or cells in an organism.

It will be appreciated that it is not necessary for a single stabilising molecule to be used, and that it is possible to utilise two or more stabilising molecules for stabilising a polypeptide. Accordingly, the term “stabilising molecule” also includes mixtures, fusions, combinations and conjugates, of atoms, molecules etc as disclosed herein. For example, a stabilising molecule may include but is not limited to: a nucleic acid combined with a polypeptide; two or more polypeptides conjugated to each other; a protein conjugated to a biologically active molecule (which may be a small molecule such as a prodrug); or a combination of a biologically active molecule with an imaging agent.

The term “stabilising molecule” may further refer to a molecule which has activity in a biological system, including, but not limited to, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, are also of use in the present invention.

As used herein, the term “system” refers to in vitro, ex vivo and in vivo systems that comprise a reversibly denatured polypeptide.

### Binding Assays

Binding of a stabilising molecule to a polypeptide may be detected using various means known in the art, including NMR spectroscopy. In a preferred embodiment the NMR involves the use of heteronuclear NMR spectroscopy. In an alternative embodiment, the NMR spectroscopy involves fluorescence anisotropy. Alternatively, the binding of a stabilising molecule to a polypeptide is detected using surface plasmon resonance or Differential Scanning Calorimetry (DSC).

All of these methods will be familiar to those skilled in the art and will be described in detail, below. Although the description may relate to stabilising molecules for p53 such as CDB3, the skilled person will be able to modify these to detect and quantify binding between a polypeptide and a stabilising molecule, or a candidate stabilising molecule.

Samples for NMR spectroscopy can be prepared using methods known to those skilled in the art. For example, samples for NMR experiments may contain ¹⁵N labeled polypeptide such as p53 core domain at a concentration of 225 μM and the corresponding stabilising molecule such as a CDB peptide in a final concentration of 22.5 mM in 150 mM KCl, 5 mM dithiothreitol (DTT), 5% D₂O in 25 mM sodium phosphate buffer pH 7.2. ¹H ¹⁵N HSQC spectra may be acquired as described in (Wong et al., 1999). In case of the p53 core domain—DNA complex, suitable DNA for use in the methods described here is the double stranded 12-mer consensus p53-binding sequence 5′-GGAACATGTTCC.

Surface plasmon resonance measurements may be performed using a BIAcore 2000 using methods familiar to those skilled in the art. For example, it may be equipped with a sensor chip SA (BIAcore AB, Uppsala, Sweden) both to screen the polypeptide for binding of stabilising molecule and to quantify the binding of the stabilising molecule to the polypeptide. For example, BIAcore may be used to screen peptides for p53 core domain binding, and to quantify the binding of p53 core domain to peptide CDB3. Biotinylated stabilising molecules such as CDB peptides may be immobilised and the binding of the polypeptide (in this case p53) can therefore be studied.

All immobilisation as well as binding measurements may be performed at 10° C. with 50 mM HEPES, pH 7.2, 5 mM DTT, as running buffer, using a sample frequency of 1 Hz. The streptavidin surface of the chip can be activated with 50 M NaOH, 1 M NaCl, in three cycles of 1 min, 20 μL/min, before the immobilization of peptides. The biotinylated peptides may be dissolved to a final concentration of 1.5-4.0 mM (in buffer as above with addition of 0.13 M NaCl) and can be immobilised at a flow rate of 5 μL/min until the level of saturation is reached. In the above Example Flow cell 1 can be used as a background for the change in bulk refractive index.

In a particular example relating to p53 and CDB3, to screen for binding to immobilised peptides, various concentrations of p53 core domain are analyzed (0.36-18 μM in buffer as above). The association phase is studied for 15 min at 10 μL/min. Bound protein is dissociated by a regeneration cycle of 1-3 min 1 M NaCl between each injection of p53 core domain.

The binding affinity of p53 core domain for immobilised CDB3 is estimated from the half saturation concentration of binding isotherm with varying concentrations of p53 core domain (0.019-0.19 μM). The binding association is measured for 5 min at 30 μL/min, 20° C., in the buffer described above (no salt added). The relative responses upon binding are plotted versus the logarithm of the p53 core domain concentrations and fitted to a two-state equation using the Kaleidagraph software (Abelbeck Software).

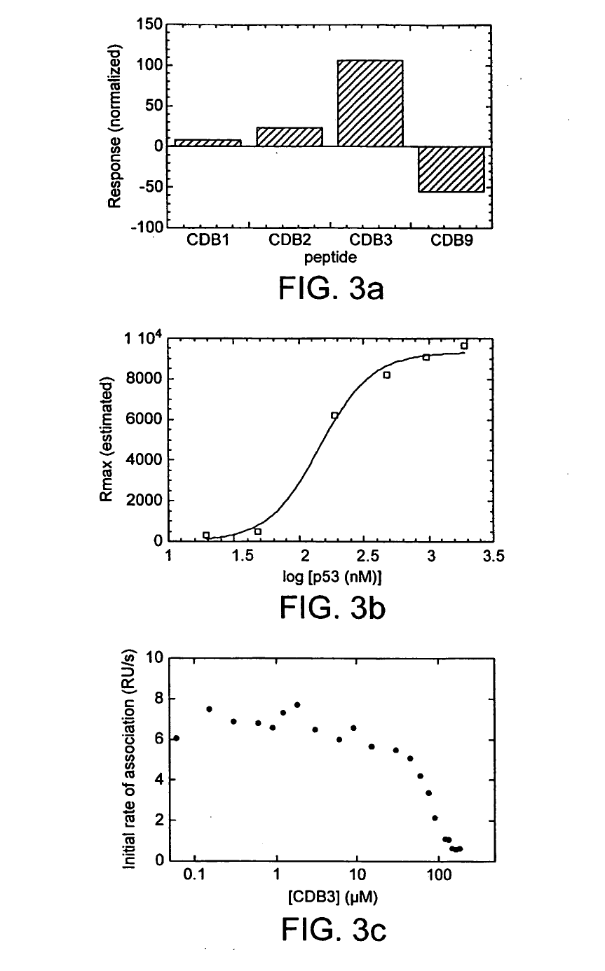

The binding affinity of soluble, unlabeled CDB3 is studied at 20° C. in buffer as described above (no salt added) using competition experiments with the BIAcore (Nieba et al., 1996). 21 samples are prepared, all containing 0.20 μM p53 core domain and various concentrations of CDB3 (0.030-120 μM). Binding data is collected in a random order of samples after 1 h of incubation at 20° C. The association phase is measured for 5 min, 30 μL/min, followed by regeneration of the surface as described above. A control sample containing protein only is analysed as every 5th sample during the experiment time as reference. The initial association rate of binding is estimated by fitting a linear equation to the first 150 s of data using the BIAevaluation 3.1 software (BIAcore AB, Uppsala, Sweden). These data (FIG. 3c), which describes the relative concentration of free p53 core domain, are analyzed according to a 1:1 binding model (Nieba et al., 1996) using Kaleidagraph. Control experiments are carried out to verify that the measured association rate of binding is proportional to concentration of p53 core domain in the range of 0.19-1.9 μM (protein only) and that the effect of increased ionic strength (due to high peptide concentration) does not significantly change the association rate (0-20 mM NaCl).

Fluorescence Anisotropy may be used to measure and/or quantify binding. For example, experiments may be performed with fluorescein-labeled CDB3 (FL-CDB3, sequence Fl-REDEDEIEW-NH₂) at 10° C. using a Perkin-Elmer LS-50b luminescence spectrofluorimeter equipped with a Hamilton microlab M dispenser controlled by laboratory software. It is not possible to make the titrations at physiological temperature, because of aggregation of the proteins. The peptide (˜5 μM, 900 μL) is dissolved in 50 mM HEPES buffer pH 7.2 which contains 5 mM DTT. Fluorescence anisotropy is measured on excitation at 480 nm (bandwidth 8 nm) and emission at 525 nm (bandwidth 2.5 nm). The free peptide has an intrinsic anisotropy value of r=0.04, which increased to a limiting value of 0.20 upon adding p53 core domain.

To determine the dissociation constant for CDB3 complexed with various p53 wild-type and mutant constructs as well as under different conditions, the following scheme is used: FL-CDB3 (900 μl, ˜5 μM) is placed in the cuvette. The appropriate p53 construct (240 μl, ˜5 μM) is placed in the Hamilton microlab M dispenser syringe. The temperature is maintained at 10° C. Additions of 3 μL of protein are titrated into the peptide solution every ˜1 min, the solution is stirred for 30 s and the anisotropy measured. The increase in anisotropy and the decrease in the total fluorescence are taken as proportional to the fluorescence contribution of the FL-CDB3-p53 complex.

Dissociation constants for the FL-CDB3-p53 complex are calculated by fitting the anisotropy and fluorescence titration curves (corrected for dilution) to a simple 1:1 equilibrium model: 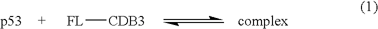 K_{d}=[p53][FL-CDB3]/[complex] (2)
[complex]=([p53]₀+[FL-CDB3]₀+K_{d}((K_{d}−[p53]₀+[FL-CDB3]₀)²+4*K_{d}*[p53]₀)^{1/2})/2 (3)

where [p53]₀ is the total protein concentration and [FL-CDB3]₀ is the total peptide concentration.

The total fluorescence at a given titration step can be described by:
Fₜₒₜₐₗ=F_{CDB3}*[FL-CDB3]/[FL-CDB3]₀+F_{complex}*[complex]/[FL-CDB3]₀ (4)
And the total anisotropy at a given time is:
Rₜₒₜₐₗ=R_{CDB3}*F_{CDB3}*[FL-CDB3]/[FL-CDB3]₀*Fₜₒₜₐₗ+R_{complex}*F_{complex}*[complex]/[FL-CDB3]₀*Fₜₒₜₐₗ

where Fₜₒₜₐₗ, and Rₜₒₜₐₗ are the total fluorescence and the total anisotropy, respectively, and F_{CDB3}, F_{complex}, R_{CDB3} and R_{complex} are the fluorescence and anisotropy values for each of the species. The data is fitted to the above equations using Marquardt algorithm and laboratory software.

Anisotropy is measured in competition experiments to study (indirectly) how CDB3 variants or Gadd45 30-mer DNA compete with the fluorescein-labeled CDB3 for the binding site of p53 core domain. The same experimental conditions described above are used, except for the slit widths (excitation 10 nm, emission 8 nm) and that the unlabeled sample is dissolved in a buffer that did not contain DTT. A stock solution of unlabeled CDB3 is titrated into a cuvette containing 900 μL 2.0 μM p53 core domain and 0.50 μM FL-CDB3 in 80 steps of 3 μL each. Competing peptide is added every 90 s, the solution is stirred for 30 s and monitoring began after 60 s. Three different stock concentrations of unlabeled CDB3 (0.26, 1.3 and 2.6 mM) and one concentration of biotinylated CDB3 (0.24 mM) are used. In case of DNA, stock solutions of 5 and 25 μM are used.

The concentrations of p53 core domain-FL-CDB3 complex and free FL-CDB3 before addition of competitor ([FP]₀ and [F]₀, respectively) are calculated using equation (3) and a given dissociation constant of 0.53 μM. The concentration of free FL-CDB3 after the n^{th} addition of stock of competitor peptide, [F]ₙ, is estimated by
[F]ₙ=(ΔRₙ/ΔR₀)*[PF]₀+[F]₀ (6)

where ΔR₀ is the change in anisotropy from FL-CDB3 only (lower limit value) to the mixture of FL-CDB3 and p53 core domain while ΔRₙ is the total change in anisotropy on the n^{th} addition. The concentration of complex between protein and unlabeled CDB3, [PU]ₙ, is determined from the total concentration of p53 core domain:
[PU]ₙ=[P]ₜₒₜₐₗ−[P]_{free,n}−[PF]ₙ (7)

When [U]ₜₒₜₐₗ (the concentration of the unlabeled peptide) is in excess over [P]ₜₒₜₐₗ (the total protein concentration), we calculated the K_{d} of the unlabeled CDB3 using equation (2).

Differential Scanning Calorimetry may also be used to detect and/or quantify binding. Differential Scanning Calorimetry may be performed using methods known to those skilled in the art. In a suitable example, DSC experiments are performed using a Microcal VP-DSC microcalorimeter (Microcal, Amherst, Mass.). Temperatures from 5 to 95° C. are scanned at a rate of 60 deg/h, using a HEPES buffer pH 7.2, 1 mM DTT, which also served for baseline measurements. Samples of wild-type and mutant p53 core domain (6-15 μM) in the presence or absence of FL-CDB3 (15-80 μM) in the above buffer are prepared and then degassed for 15 min prior to each experiment. A pressure of 25 psi (1.56 atm) is applied to the cell. The data is analysed using Origin software (Microcal).

### Peptides, Polypeptides and Proteins

The methods described here are suitable for stabilising the native form of polypeptides. Preferably, the stabilising molecule comprises a polypeptide. As used in this document, the terms “peptide”, “polypeptide” and “protein” are synonymous with each other.

The term “peptide” in the context of this document includes two or more amino acids linked together by a peptide bond. Typically, they have more than 5, 10 or 20 amino acids and can be any length up to 600 amino acids. In a preferred embodiment, the peptide has less than 200 amino acids, in a particularly preferred embodiment it has less than 100 amino acids, in a preferred embodiment still it has less than 50 amino acids. In a still further preferred embodiment it has less than 20 amino acids. In a most preferred embodiment it has less than 10 amino acids. A polypeptide or protein includes single-chain polypeptide molecules as well as multiple-polypeptide complexes where individual constituent polypeptides are linked by covalent or non-covalent means.

One skilled in the art will appreciate that the particular amino acid composition of a stabilising molecule which is a peptide will depend on the protein to which it is to be bound. Amino acids may be naturally occurring or synthetic. Those skilled in the art will be aware of suitable sources of amino acids.

A polypeptide (including a peptide stabilising molecule) may be generated using synthetic methods, which will be known to those skilled in the art. Alternatively, it may be generated from naturally occurring or synthetic proteins, and/or polypeptides, and/or peptides. Degradation of the proteins, polypeptides or peptides may be performed by enzymatic and/or chemical digestion, using methods familiar to those skilled in the art. Those skilled will be aware of other suitable methods of degradation.

The term “peptide” in the context of this document, also includes within its scope, derivatives and variants thereof, as herein described.

Examples of derivatives include peptides which have undergone post-translational modifications such as the addition of phosphoryl groups. It may also include the addition of one or more of the ligands selected from the group consisting of: phosphate, amine, amide, sulphate, sulphide, biotin, a fluorophore, and a chromophore. One skilled in the art will appreciate that this list is not intended to be exhaustive. In a preferred embodiment of this aspect, a stabilising molecule which is a peptide is derivativised using a fluorophore. In an especially preferred embodiment, the fluorophore is fluorescein.

The terms “variant” or “derivative” in relation to the amino acid described here includes any substitution of, variation of, modification of, replacement of, deletion of or addition of one (or more) amino acids from or to the sequence.

Variants of the peptides described here are likely to comprise conservative amino acid substitutions. Conservative substitutions may be defined, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

### Peptide Synthesis

Peptides may be synthesised using methods known to those skilled in the art. A typical procedure is detailed below:

Peptides may be synthesized using a 432A Synergy peptide synthesizer (Applied Biosystems (ABI)). Protected amino acid derivatives, reagents and solvents may be purchased from ABI, except for Fmoc-Ser(PO(OBzl)OH)-OH, which can be purchased from NOVAbiochem. Standard Fmoc chemistry can be employed, with coupling agents HBTU/HOBt. The peptides can be cleaved from the resin using a mixture of trifluoroacetic acid:Triisopropylsilane:water 90:5:5, precipitated in cold ethyl ether, washed 3 times with cold ethyl ether, dissolved in water or in a mixture of water:acetonitrile 1:1 and lyophilized.

The peptides can be purified using reverse-phase HPLC (Waters 600 equipped with a 996 PDA detector). The column may be a preparative reverse phase C8 column (Vydac) and the gradient is 100% A to 100% B in 35 min (A=0.1% TFA in water, B=31 95% acetonitrile, 5% water, 0.1% TFA). The purified peptides are characterized by MALDI-TOF MS and had the expected M_{w}.

For biotinylated peptides, the biotin may be coupled to the N-terminus through its carboxylic acid group during the solid-phase synthesis. The same conditions may be applied for the biotin coupling as for the coupling of the protected amino acids, except that it is repeated twice in some cases. Proteins and peptides may also be purchased commercially; for example, fluorescein-labeled CDB3 is purchased from Dr Graham Bloomberg (University of Bristol, UK).

Methods of protein and polypeptide synthesis are known in the art and are described in for example, Maniatis et al. For example, proteins such as human p53 core domain wild-type and mutants (residues 94-312) and human tetrameric p53 (residues 94-360) may be cloned, expressed and purified using methods familiar to those skilled in the art, in particular those described previously (Bullock et al., 1997). ¹⁵N-labelled human p53 core domain may be produced as described previously (Wong et al., 1999).

### Uses of Stabilising Molecules

We further describe a composition comprising at least one or more stabilising molecules and a pharmaceutically acceptable carrier, diluent or exipient.

Stabilising molecules, which are preferably peptides, and compositions described here may be employed for in vivo therapeutic and prophylactic applications, in vitro and in vivo diagnostic applications, in vitro assay and reagent applications, and the like.

Therapeutic and prophylactic uses of the stabilising molecules and compositions described here involve the administration of the above to a recipient mammal, such as a human.

The term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the selected stabilising molecules or peptides or compositions in protecting against or treating the disease are available and will be familiar to those in the art.

Generally, the stabilising molecules, peptides or compositions will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack, Remington's Pharmaceutical Sciences, 16^{th} ed. (1982)).

The selected stabilising molecules described here may be used as separately administered compositions or in conjunction with other agents. These can include various immunotherapeutic drugs, such as cylcosporine, methotrexate, adriamycin or cisplatinum, and immunotoxins or in conjunction with radiotherapy or radioisotopes or other types of radiation. Pharmaceutical compositions can include “cocktails” of various agents.

The route of administration of pharmaceutical compositions may be any of those commonly known to those of ordinary skill in the art. For therapy, including without limitation immunotherapy, the selected stabilising molecules or compositions can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. The peptides may also be administered by expression from a DNA or RNA-based vector, including viral vectors capable of transducing the cells. For example, retroviral, lentiviral or poxviral vectors may be used to transduce cells with nucleic acid encoding the CDB3 peptide. As an alternative, direct injection of the nucleic acid can be employed.

Alternatively, or in addition, chemical reagents may be employed in order to facilitate the uptake of the peptide or nucleic acid encoding the peptide into cells. Suitable chemical reagents include calcium phosphate and DEAE-dextran for nucleic acids; and Lipofectamine™, liposome-based delivery systems, fusions with peptides such as viral fusogenic peptides, nuclear transfer peptides such as VP22 and penetratin, and the like, for the delivery of peptides. Those skilled in the art will appreciate that this list is not intended to be exhaustive.

The present inventors have found that the peptide CDB3, particularly in fluorescein-labelled form, is able to penetrate inside the cells by itself, although the efficiency of the delivery is enhanced by the use of chemical reagents such as Lipofectamine™. Furthermore, the present inventors have found that there is a pronounced nuclear localisation of CDB3 in cells that express wild-type p53 or the severely compromised mutant R175H than there is in cells lacking p53. As p53 normally exerts its effects in the nucleus, then this suggests that CDB3 forms a complex with p53 and is subsequently transported into the nucleus.

The selected stabilising molecules, peptides or compositions can be lyophilised for storage and reconstituted in a suitable carrier prior to use. Known lyophilisation and reconstitution techniques can be employed. It will be appreciated by those skilled in the art that lyophilisation and reconstitution can lead to varying degrees of functional activity loss and that use levels may have to be adjusted upward to compensate.

The compositions containing the stabilising molecules or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide or other stabilising molecule per kilogram of body weight, with doses of 0.05 to 2.0 mg/kg/dose being more commonly used. For prophylactic applications, compositions containing the present selected stabilising molecules or cocktails thereof may also be administered in similar or slightly lower dosages.

Stabilising molecules and/or compositions can be used in the treatment of any disease where errors in protein conformation, folding and aggregation contribute to the disease. Examples include cancer, cystic fibrosis and neuro-degeneration. One skilled in the art will appreciate that this list is not intended to be exhaustive. The efficacy of disease treatment with a stabilising molecule as described herein can be determined by one of skill in the art on the basis of a modulation of one or more symptoms or parameters associated with the disease phenotype (e.g., a favorable change of at least 10% in a disease parameter or symptom, or, alternatively, by at least one point on a clinically accepted scale used to assess symptoms or severity of a particular disease). In a particularly preferred embodiment, the disease is cancer. For tumors, a slowing of tumor growth rate by at least 10% after treatment is evidence of effective treatment, as are a halt in tumor growth, and regression or complete disappearance of the tumor. Methods of detecting tumors and neoplasias and methods of monitoring their growth are well known to those of skill in the art.

### Stabilisation of P53

In a highly preferred embodiment, we provide a stabilising molecule capable of stabilising the native state of a p53 polypeptide.

This preferred stabilising molecule is a polypeptide, and comprises a 9 amino-acid residue peptide, having the sequence REDEDEIEW-NH₂. This peptide is referred to as CDB3. We also provide a fluorescein-labeled derivative of CDB3 (FL-CDB3) which can bind and stabilise p53 core domain.

CDB3 is derived from the p53 binding polypeptide 53BP2 and consists of residues 490-498 of that protein. Resides 490-498 constitute one of the p53 binding loops in the protein. The most striking properties of FL-CDB3 are its abilities to: (1) stabilise p53 core domain, as shown by raising its apparent melting temperature, and (2) induce refolding of reversibly denatured p53 core domain. Thus, a small peptide can stabilise p53 core domain simply by binding its native state but not the denatured state and shifting the equilibrium towards the native form.

### Polypeptide Target Site

It may be necessary to identify the binding site for a stabilising molecule within the polypeptide to be stabilised. This may be done in various ways as known in the art.

Structural characterisation of the CDB3 binding site within the p53 core domain is a key point, since this site might serve as a specific target for core domain stabilising molecules. The CDB3 binding site, as mapped by NMR chemical shift analysis, is situated at the edge of the DNA-binding site and consists of three structural elements (loop 1, helix 2 and the edge of strand 8) which are remote sequentially but close spatially. The advantage of this site as a general target for p53-stabilising molecules is its location in proximity to the DNA binding site, enabling a local stabilising effect in that site. Indeed, chemical shift data shows the difference between the effects of DNA binding and CDB3 binding. CDB3 binding generates a strong localized effect on the DNA-binding site within p53 core domain, while the chemical shift pattern upon DNA binding is significantly different, with shifts that are not as localized but are rather spread throughout the whole protein structure.

An intriguing observation is that CDB3 does not bind p53 core domain in the same location as the parent loop in the 53BP2 protein (Gorina and Pavletich, 1996). The original 53BP2 loop binds the core domain between helix 2 and loop 3, with Trp498 of 53BP2 making contacts mainly with loop 3 of p53, and the carboxylic acid side chains of 53BP2 making contacts with p53 Arg273 (a DNA-binding residue located in strand 10, close to helix 2) (Gorina and Pavletich, 1996). The CDB3 binding site might also be an additional binding site for 53BP2, and the two alternative binding sites might have a regulatory role. The observation that CDB3 and the original 53BP2 loop bind p53 at different sites might also be explained by the partly electrostatic nature of the interaction. Owing to its high negative charge, CDB3 as a free peptide might act partly as a negatively charged “DNA-mimic”, which binds the positively charged surface of the DNA-binding site.

### Rescue of p53 Core Domain Mutants

CDB3 is found to bind two p53 core domain hot-spot mutants: G245S, which is weakly destabilised (Bullock et al., 2000), and R249S, which is distorted in the DNA binding region (Bullock et al., 2000; Wong et al., 1999). FL-CDB3 affinity to the G245S mutant, which is folded almost as the wild-type (Bullock et al., 2000), is the same as for the wild type. Binding to the R249S mutant, which is more destabilised, is weaker (but still in the low micromolar range). In addition CDB3 binds to a particular p53 core mutant (195T) which is highly destabilised. The mutation in this mutant is not in one of the typical oncogenic hot-spots.

The observation that CDB3 binds mutants raises the possibility that such compounds can be used to rescue such mutants by stabilising them. Since their general mechanism of action is simply binding the native state and shifting the equilibrium, CDB3-like compounds could be used for the rescue of weakly destabilised (e.g. G245S) and globally unfolded (e.g. V143A) mutants that are unable to bind DNA (see below). The application of CDB3 for the rescue of locally distorted mutants, such as R249S, depends on the specific binding mode of the peptide as well on the specific distortion caused by the mutation. In general, locally distorted mutants require more specific molecules, which alter the conformation near the distorted site. We demonstrate that it is possible for R249S: FL-CDB3 stabilises it since it binds in proximity to the distortion site (near loops 2 and 3 in the DNA binding site and see Wong et al., 1999). FL-CDB3, which binds the DNA binding site at its edge, might contribute to a local conformational change at this site of distortion.

The mode of action of CDB3 is different from that of the previously reported p53 C-terminal peptides. CDB3 stabilises p53 by binding its native but not its denatured state, while the C-terminal peptides specifically regulate the activity and the DNA binding of p53 core domain (Abarzua et al., 1996; Hupp et al., 1995; Selivanova et al., 1997; Selivanova et al., 1999). CDB3, and especially its labeled derivative FL-CDB3, are lead compounds, and they can be used as a basis for the future design of peptides and small molecules that have a larger stabilising effect on p53 core domain. Peptides such as CDB3 cannot be used to rescue DNA contact mutants. Other strategies, which involve introduction of residues or small molecules that contribute the missing interactions, should be used for rescue of these mutants.

### EXAMPLES

The invention is further described, for the purposes of illustration only, in the following examples which are in no way limiting of the invention.

These examples relate to the isolation and identification of a stabilising molecule CDB3, which is capable of binding the tumour suppressor protein p53 near its DNA binding site, and stabilising the native form of the protein.

The inherent drawback of using a natural binding site for a drug is that it competes with the natural ligand. Thus, it might be thought that the competition between DNA and CDB3 peptide would preclude it from being of use as a lead compound. But this need not be so. Since the binding of DNA itself stabilises p53 core domain, and it binds very tightly, stabilisation by a peptide such as CDB3 is needed only for mutants where DNA binding is impaired because mutant p53 is in denatured conformation. Once the protein has bound DNA, the peptide is not needed any more.

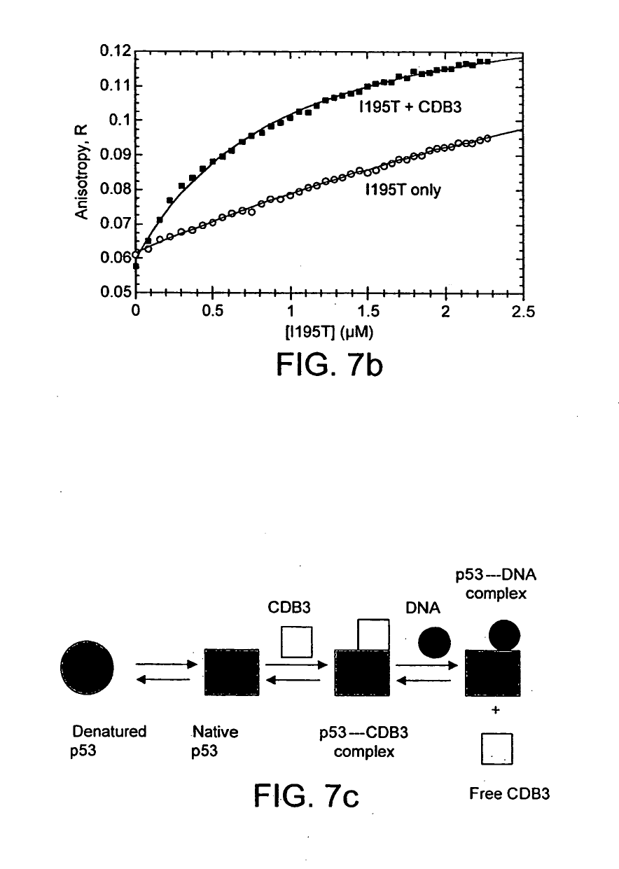

The ability of CDB3 to induce refolding of p53 core domain, together with the observation that DNA can displace it from p53, led us to propose the a “chaperone” mechanism for rescuing a denatured oncogenic protein (FIG. 7c): CDB3 binds only the native state of the oncogenic protein which is able to bind DNA, probably immediately on biosynthesis, and therefore shifts the equilibrium towards the native state. Then DNA can bind the protein, displacing the peptide, which is free again to bind another protein molecule. Further, the peptide binds equally well to a monomer of p53 and the tetramer, but DNA binds far more tightly to the tetramer because of cooperativity (unpublished), thus allowing DNA to displace the drug more easily.

### Example 1

### Design of Potential p53 Core Domain Binding Peptides

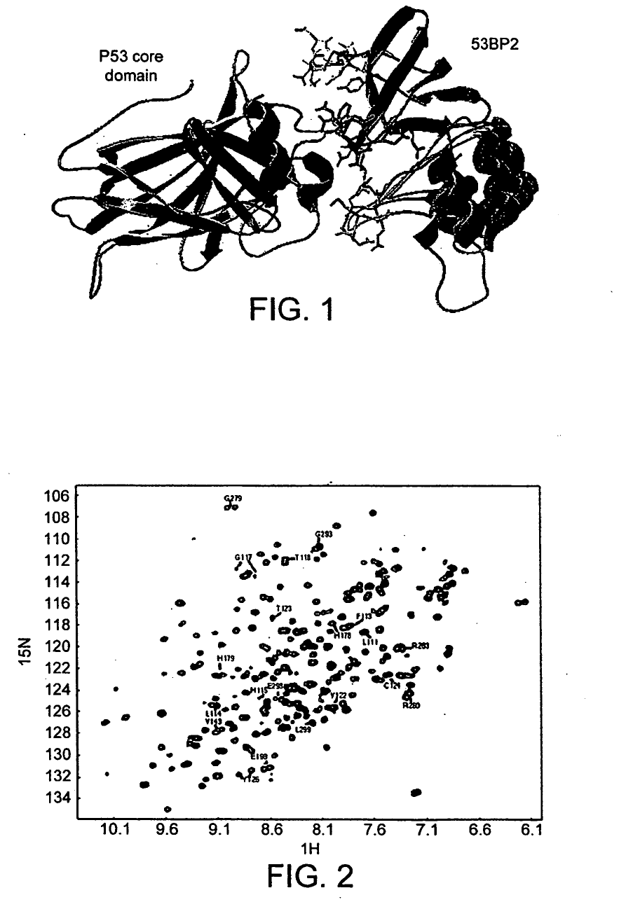

Peptides that bind the native state of p53 core domain can be derived from p53 binding proteins. A rare example of a complex of a protein bound to p53 that has been solved at high resolution is the p53 core domain-53BP2 complex (Gorina and Pavletich, 1996). 53BP2 is a p53 binding protein (Iwabuchi et al., 1994) that enhances p53-mediated transactivation, impedes cell cycle progression and induces apoptosis (Iwabuchi et al., 1998; Lopez et al., 2000). 53BP2 binds p53 core domain in its DNA binding site, with three of its loops making the contacts with p53 (Gorina and Pavletich, 1996) (FIG. 1). Three peptides corresponding to these three loops are synthesized and tested (Core Domain Binding (CDB)1-3, see Table 1).

A second potential source for core domain binding peptides are sequences within p53 itself that bind the core domain and regulate its activity. Two such regions within p53 are the C-terminal domain (amino acids 363-393) (Bayle et al, 1995) and the proline-rich domain (amino acids 54-94) (Muller-Tiemann et al., 1998). Several overlapping peptides corresponding to both regions are synthesized (CDB4, 7-10 in Table 1). Since Ser378 within the C-terminal domain is known to undergo phosphorylation (Takenaka et al., 1995), phosphopeptides derived from this region are synthesized as well (CDB5, 6 in Table 1). The C-terminal and the proline-rich domains can bind the core domain only in presence of each other (Kim et al., 1999), and thus a fusion peptide between these domains is also designed (CDB11 in Table 1).

Screening of the CDB Peptides for Binding p53 Core Domain

Initial screening for binding of the peptides to p53 core domain are made using heteronuclear NMR spectroscopy to monitor any changes in the backbone ¹H and ¹⁵N resonances of ¹⁵N labelled p53 core domain (Wong et al., 1999). Chemical shift changes are observed only with CDB2 and CDB3, implicating binding of only these peptides to p53 core domain (FIG. 2).

To estimate the peptides' affinity for p53 core domain, surface plasmon resonance can be used. Peptides CDB1, 2, 3, 9 and 11 are re-synthesized with a biotin label attached to their N-terminus. The biotinylated peptides are immobilized onto a streptavidin (SA) sensor chip, p53 core domain (7.2 μM) is injected, and the binding is monitored using a BIAcore instrument. The relative response for the different peptides, corrected for the control flow channel (SA chip with no peptide immobilized), is shown in FIG. 3a. p53 core domain had the tightest binding to CDB3, in good agreement with the NMR data which showed that p53 core domain bound CDB3 better than CDB2. There is no significant binding to CDB 1 or CDB9.

### Example 2

### Characterization of CDB3-p53 Core Domain Binding

Surface plasmon resonance can be used to measure the CDB3-p53 core domain binding constant quantitatively. Biotinylated CDB3 is immobilized on a SA sensor chip, and p53 core domain (0.02-2 μM) is injected (FIG. 3b). The concentration of p53 core domain for 50% binding is estimated to be 200 nM.

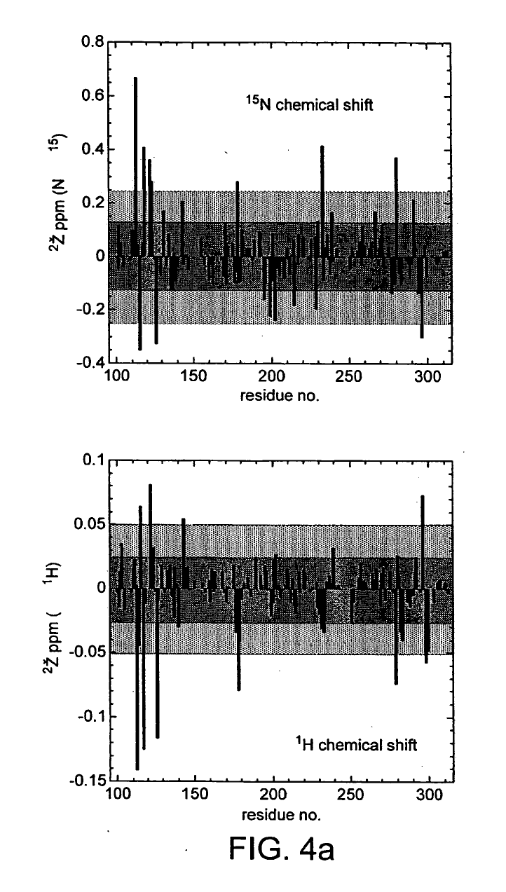

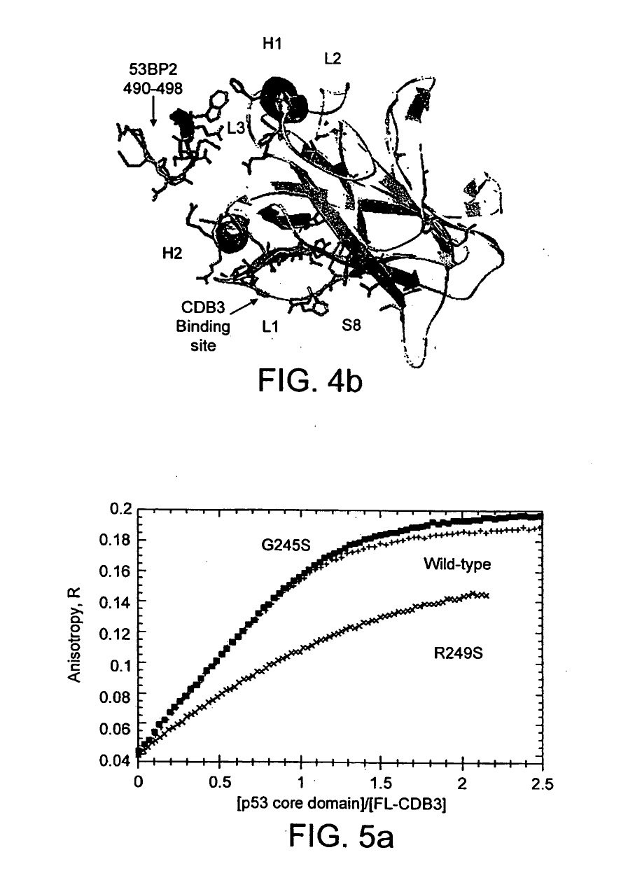

Chemical shift differences between the spectra of the bound and unbound p53 core domain are used to identify the site in p53 core domain where CDB3 bound. Changes of backbone ¹H—¹⁵N resonances for each residue between the bound and the unbound states are shown in FIG. 4a. Chemical shift changes are found mainly in loop 1, helix 2 and strand 8 (colour coded blue and purple in FIG. 4b), which are located at one edge of the DNA binding site. Chemical shift changes in presence of CDB3 are also observed for two residues in helix 1, but these do not define a binding site but are probably due to a weak non-specific interaction. It appears that CDB3, as a free peptide (colour coded red in FIG. 4b), bound p53 core domain in a different location from that of the original loop within 53BP2 (Gorina and Pavletich, 1996). It binds loop 1, strand 8 and helix 2 that are at the edge of the DNA binding site, rather than its original place in the middle of the DNA-binding site that consists of loop 3 and the other side of helix 2.

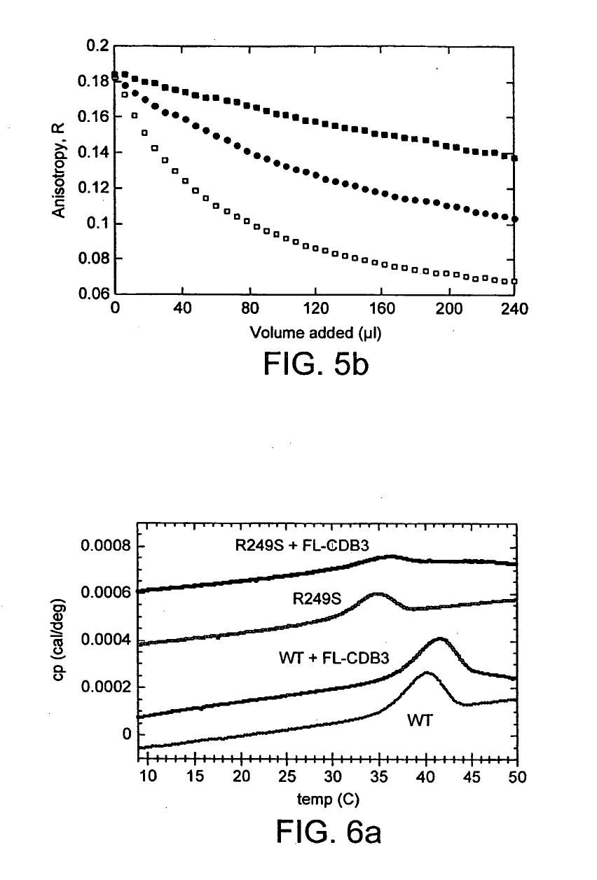

Fluorescence anisotropy titrations may be used to determine the dissociation constant for the p53 core domain-CDB3 interaction at 10° C. p53 core domain is titrated into fluorescein-labeled CDB3 (FL-CDB3) and changes in anisotropy of the labeled peptide (FIG. 5a) as well as the total fluorescence at 525 nm are monitored. The initial anisotropy value for the labeled peptide is 0.04, and the limiting value for the FL-CDB3-p53 core domain complex is 0.20. The binding curve is fitted to a 1:1 simple equilibrium model, and the K_{d} is found to be 0.53±0.09 μM (Table 2). In order to confirm that CDB3 binds tetrameric p53, and not only isolated core domain (residues 94-312), K_{d} for the binding to the tetrameric p53 construct (p53 94-360) is determined in the same way and is found to be 0.77±0.09 μM.

### Example 3

### Binding of Fluorescein-Labeled Peptides

To determine whether attaching different labels (fluorescein and biotin) to CDB3 N-terminus alters K_{d}, the dissociation constants for the unlabeled peptide in competition experiments by two independent methods can be measured: competition BIAcore (FIG. 3c); and anisotropy (FIG. 5b). The unlabeled peptide had a K_{d} of 37 μM (Table 3). Biotinylation of the N-terminus improved the affinity (compared to the unlabeled peptide) three-fold for solution measurements (K_{d}=12 μM, see FIG. 5b and Table 3) and even more for the immobilised sample—BIAcore assays (K_{d}=0.2 μM, see FIG. 3b). Perhaps the unsubstituted peptide is bound more weakly because of the electrostatic repulsion between the positive charge on its N-terminal-amino group and the positively charged protein surface. Alternatively, fluorescein itself could improve the affinity.

FL-CDB3 Stabilises p53 Core Domain and Raises its Apparent Tₘ

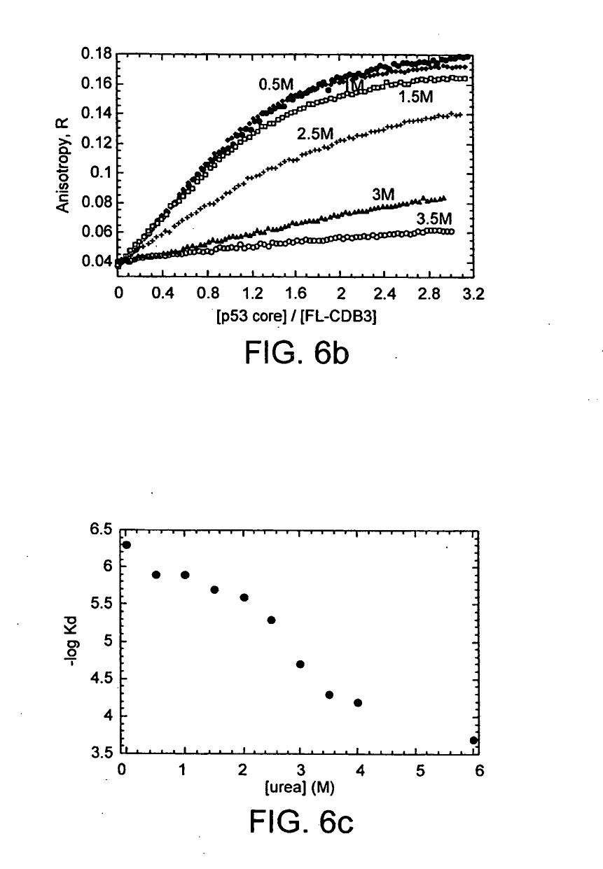

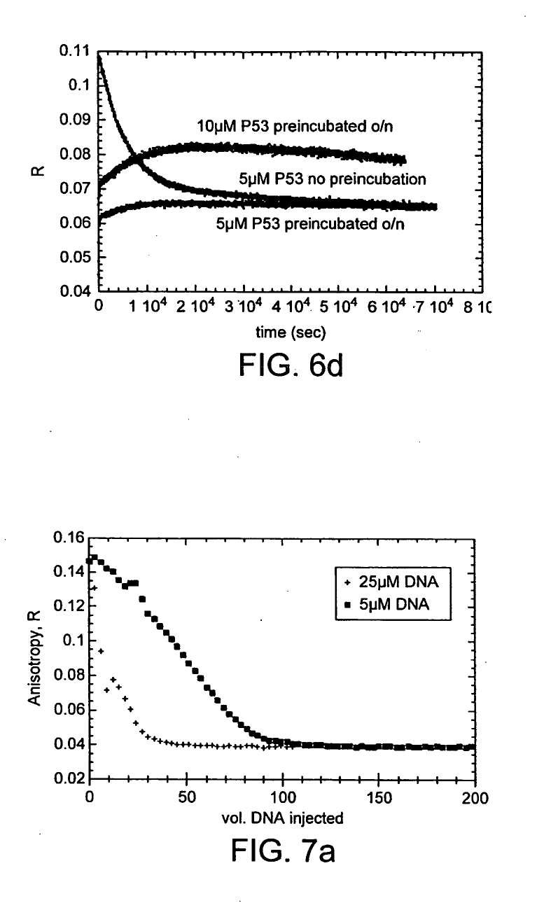

Differential scanning calorimetry (DSC) is used to detect stabilisation of p53 core domain by FL-CDB3. The thermal denaturation of p53 core domain is irreversible, and thus only an apparent melting temperature (Tₘ) can be determined (Bullock et al., 1997), but increase in stability can be correlated with increase in the apparent Tₘ. All DSC measurements are carried out in HEPES buffer pH 7.2, 1 mM DTT. Under these conditions the apparent Tₘ of wild-type p53 core domain is 40.1° C. Tₘ increased by 1.5 degrees in presence of the peptide FL-CDB3 (FIG. 6a), showing a stabilising effect of the peptide. The apparent Tₘ of the mutant R249S is 34.9° C., which increased to 35.9° C. in presence of the peptide. The signal for the R249S mutant is weaker due to increased aggregation of the protein. The unlabeled CDB3 at the same concentrations did not induce a shift in _{Tm} (not shown).

### Example 4

### FL-CDB3 Binds the Native, and Not the Denatured, State of p53 Core Domain

FL-CDB3, as a peptide that stabilises p53 core domain, binds the native, but not the denatured state. It should bind “wild-type-like” folded and stable mutants with a similar affinity to that of the wild type, but bind partly unfolded and distorted mutants with a lower affinity. The binding of FL-CDB3 to two p53 core domain mutants is measured: to G245S, which is 95% folded at 37° C. and is destabilised by 1.21 kcal/mol at 10° C.; and to R249S, which is 85% folded at 37° C., is distorted and is destabilised by 1.92 kcal/mol at 10° C. (Bullock et al., 1997; Bullock et al., 2000). At 10° C. both mutants are expected to be in a native-like conformation. K_{d} values at this temperature, from fluorescence anisotropy (see FIG. 5a), are 0.57±0.09 μM for G245S and 3.3±0.5 μM for R249S, indicating weaker binding of the more destabilised mutant.

To confirm that FL-CDB3 binds the native and not the denatured state of p53 core domain, we repeated the fluorescence anisotropy titrations in presence of increasing urea concentrations (FIG. 6b). p53 core domain is incubated overnight in different urea concentrations, and is titrated into FL-CDB3 which is dissolved in the same urea concentration. A plot of log K_{d} vs. urea concentration (FIG. 6c) showed that the binding weakened with increasing urea concentrations, where more of the protein becomes unfolded. Quantitative analysis indicated that urea also weakened the binding of the peptide to the native p53 core domain (not shown).

### Example 5

### FL-CDB3 Induces Refolding of p53 Core Domain

The ability of CDB3 to refold a partly denatured p53 core domain is determined using fluorescence anisotropy. p53 core domain is incubated overnight at 10° C. in 3 M urea, under which conditions it is predominantly denatured. Then it is mixed with FL-CDB3 in 3 M urea, and the changes in anisotropy over time are monitored (FIG. 6d). The initial anisotropy value for the labeled peptide is 0.04. Upon mixing the peptide with p53 core domain a rapid binding event takes place, leading to the formation of a FL-CDB3-p53 core domain complex. The anisotropy values for the complex following preincubation overnight with 3 M urea are 0.06-0.07, far below the limiting anisotropy value for the bound complex at these FL-CDB3 and p53 core domain concentrations (no urea), which is 0.17 (estimated from FIG. 5a), because under these conditions most of the protein is denatured and did not bind the peptide. There is an increase in the anisotropy over time, as the peptide induced protein refolding by mass action (FIG. 6d). On mixing p53 core domain and CDB3 (5 μM each) with 3 M urea without preincubation overnight, unfolding took place reaching the same endpoint. Overall, CDB3 induced refolding of p53 core domain and in its presence the equilibrium shifted towards the native state. Urea weakened the binding to the native structure so that the stabilising effects in 3 M urea are not as pronounced as they would be in water alone.

### Example 6

### DNA Competes with FL-CDB3 for p53 Core Domain Binding

From the NMR data (FIG. 4) it seems that CDB3 binds p53 core domain at the edge of the DNA binding site, suggesting at least a partial overlap between the two binding sites. We have measured the competition between the binding of FL-CDB3 and Gadd45 DNA to p53 core domain using fluorescence anisotropy. DNA (5-25 μM) displaced the peptide completely from the binding site, indicating overlap between the DNA and peptide binding sites (FIG. 7a). When p53 core domain is titrated into a mixture of CDB3 and DNA, only binding to DNA but not binding to the peptide could be determined (not shown).

To get more structural information regarding CDB3 versus DNA binding, we used heteronuclear NMR spectroscopy. An HSQC spectrum of p53 core domain in presence of 12-mer consensus DNA sequence is taken, and the DNA site is mapped using chemical shift analysis, exactly as done for the CDB3 binding site. Chemical shifts in presence of the DNA are distributed throughout the whole protein, and can be found in the DNA binding site as well as in the beta-sandwich. Significant shifts can be observed, for example, for residues in loop L1 (S121), strand S10 (R273, R274), L3 (M237, S241), L2 (C176), S4 (A159), S6 and S7 (R202, V216, Y220), the hinge between S9 and S10 (L257, D259), and helix H2. Overall, there is a significant difference in the chemical shift pattern upon binding CDB3 and DNA. The CDB3 site is well localized to the H2-L1-S8 region, especially in L1, while the DNA binding affects the conformation of different regions throughout the protein.

### Example 7

### CDB3 Restored Sequence-Specific DNA Binding to the Highly Destabilized p53 Mutant I195T

We tested whether CDB3 can restore sequence-specific DNA binding activity to p53 core domain mutants by observing its effect on the β-sandwich mutant I195T, which is highly destabilized by 4.1 kcal/mol (Bullock et al., 2000) and has poor binding affinity. I195T (10 μM) was incubated for 1 h at 10° C. in presence of CDB3 (100 μM) (or its absence) and titrated into fluorescein-labelled Gadd45 DNA in presence of the same peptide concentration. In the absence of peptide, I195T bound Gadd45 DNA with K_{d}=6 μM (FIG. 7b). After incubation with CDB3, the binding improved six fold, and K_{d} was 1 μM, which is close to the value of 0.8 μM for wild type. As expected, CDB3 did not affect DNA binding of the completely native wild-type p53 core domain (not shown).

To confirm that the restoration of DNA binding is sequence-specific, we repeated the experiments with the random double stranded DNA sequence fluorescein-AATATGGTTTGAA TAAAGAGTAAAGATTTG. Binding of I195T to this sequence was very weak, and was not improved, but rather inhibited, by the peptide (not shown).

### Example 8

General Methods

The general methods used in the examples set forth below are described in detail in Bykov et al., Nature Med 8, 282-288 (2002) which is herein incorporated by reference.

Cell Lines

The following cell lines were used in the experiments described in the following examples:

H1299 lung carcinoma, which had both p53 alleles deleted; H1299-His175, which was H1299 transfected with R175H mutant; Saos-2, osteosarcoma, both p53 alleles deleted; Saos-2-His273, transfected with R273H mutant; HCT116p53+/+, which has wild-type 53 (as well as a high level of Mdm2 and ARF deleted); and HCTp53−/−, in which both p53 alleles were deleted by homologous recombination.

### Example 9

### Distribution of FL-CD 83 in Cell After Treatment with FLCD 83 Peptide for 24 Hrs

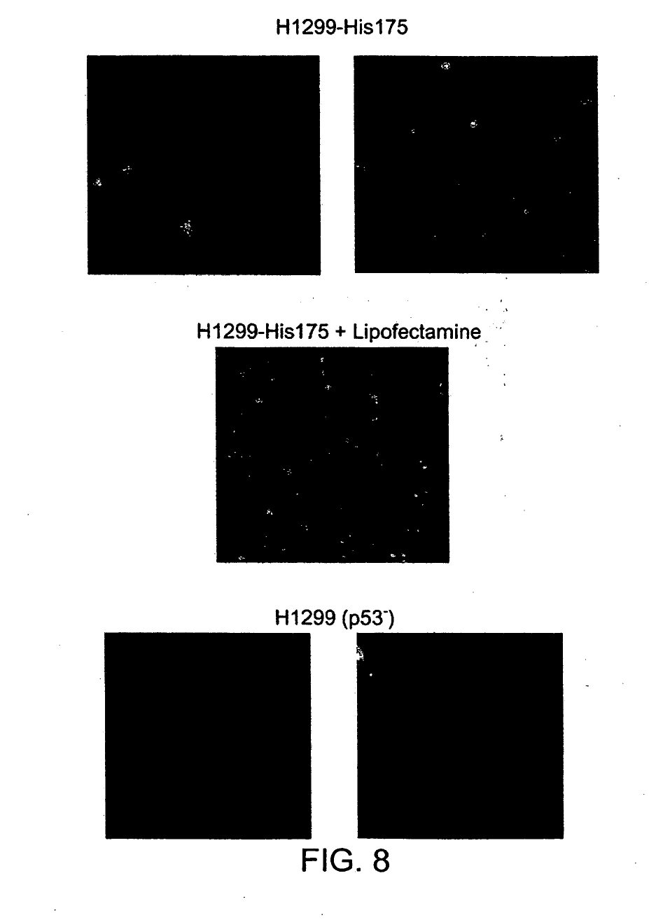

The results can be seen in FIG. 8. Details of the methods used are described in Bykov et al., Nature Med 8, 282-288 (2002).

FIG. 8 shows the distribution of FL-CDB3 in cells after treatment with peptide for 24 h. The nuclei are visible in blue (staining with Hoechst), the peptide is green. Top left: H1299 cells containing p53 R 175H. FL-CDB3 was localised in nuclei and large deposits could be seen in a nucleolus. Top right: cytoplasmic distribution was also observed in some cases. Middle: after combined delivery with Lipofectamine 2000™, the peptide was located in the cytoplasm, although some nuclear fraction was present as well. Bottom left and right: distribution of the peptide in parental p53-null H1299 cells. It appears that in p53 null cells peptide is localised mostly in cytoplasm (H1299), although in some cells nucleolar localisation is also evident (H12991-1). The peptide remained visible for at least 48 h.

In conclusion, the peptide FL-CDB3 is able to penetrate inside the cells by itself, although the efficiency of delivery could be enhanced by Lipofectamine (FIG. 8). Importantly, there is a much more pronounced nuclear localisation of FL-CDB3 in cells that express wild-type p53 or the severely compromised mutant R175H than there is in cells lacking p53. p53 normally exerts its activity in the nucleus.

### Example 10

### Detection of Induced Protein Expression by Western Blots After 24 H Incubation with FL-CDB3

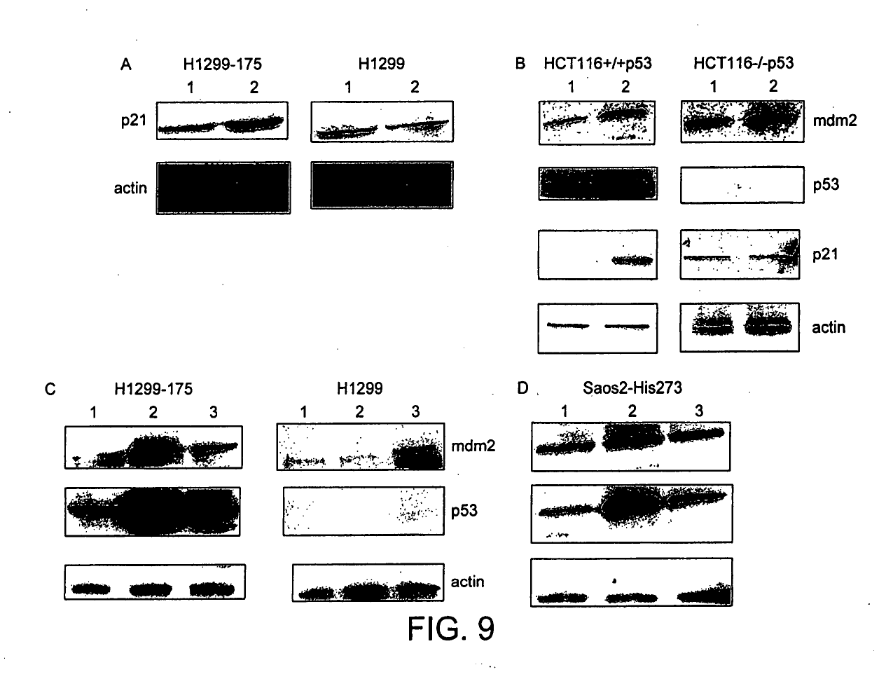

The results can be seen in FIG. 9.

Frames A, C, and D: Treatment with FL-CDB3 restored the ability of p53 mutants His175 and His273 to activate the transcription of endogenous genes p21 and Mdm-2. Lung carcinoma cells H1299 transfected with His175 p53 mutant and parental nontransfected cells were treated with the amounts of peptide indicated below, incubated for 24 h and tested for p53, p21, and Mdm-2 protein expression. The levels of actin show the equal loading of protein. Notably, mutant p53 levels were remarkably increased. B: Treatment with FL-CDB3 induces wtp53 in colon carcinoma HCT1 16 cells and activates expression of Mdm-2 and p21. No induction of p21 nor Mdm-2 was observed in the absence of p53 expression in HCTp53−/− cells. For A and B: Lane 1 was the control with no FL-CDB3; Lane 2 was 24 h post treatment with 10 μg/mL FL-CDB3. For C and D, Lane 1 was the control (no FL-CDB3); Lane 2, 10 μg/mL FL-CDB3; and Lane 3, 1 μg/mL FL-CDB3. The treatment with peptide was performed either with or without Lipofectamine. All the data presented here were obtained after treatment without Lipofectamine, except frames C and D. The induction of p53 target genes in C and D is seen to be dependent on the concentration of FL-CDB3.

Overall, the peptide induces endogenous p53 target genes p21 and Mdm-2 in a p53-dependent manner (FIG. 9). Two mutants were tested, H273 and H175. Surprisingly, the transcriptional activity of both of them was reactivated. All experiments were repeated at least three times. Interestingly, the transcriptional function of wild-type p53 is also activated. The levels of wild-type and mutant p53 protein were considerably raised.

### Example 11

### FACS Analysis of Effects of FL-CDB3 on Cell Cycle

Tumour cells were treated with 10 μg/mL of peptide and analysed the cell cycle distribution and cell death (as subG1 fraction) 24 h post treatment using FACS analysis. The left hand side of each pair of panels is the control without FL-CDB3. In one experiment, the percentage of dead cells was determined by trypan blue exclusion: the number of dead cells in H1299-His175 cells before treatment was 5%, after treatment, 37%; in control H1299 (p53^{˜}), before 3%, after treatment 11%; in Saos-2-His273 cells, before 3%, after 28%; in control Saos-2(p53^{˜}), before treatment 3%, after 13%.

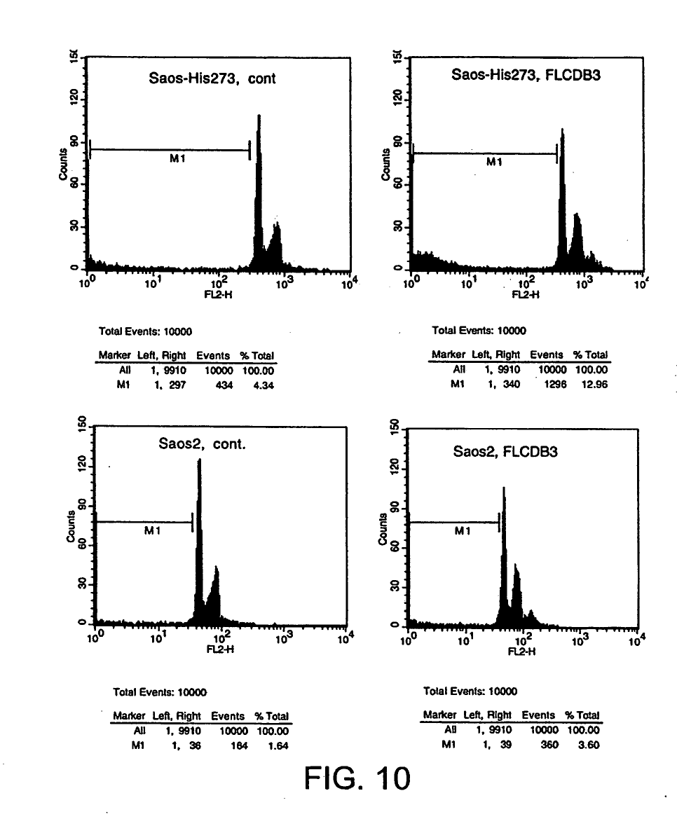

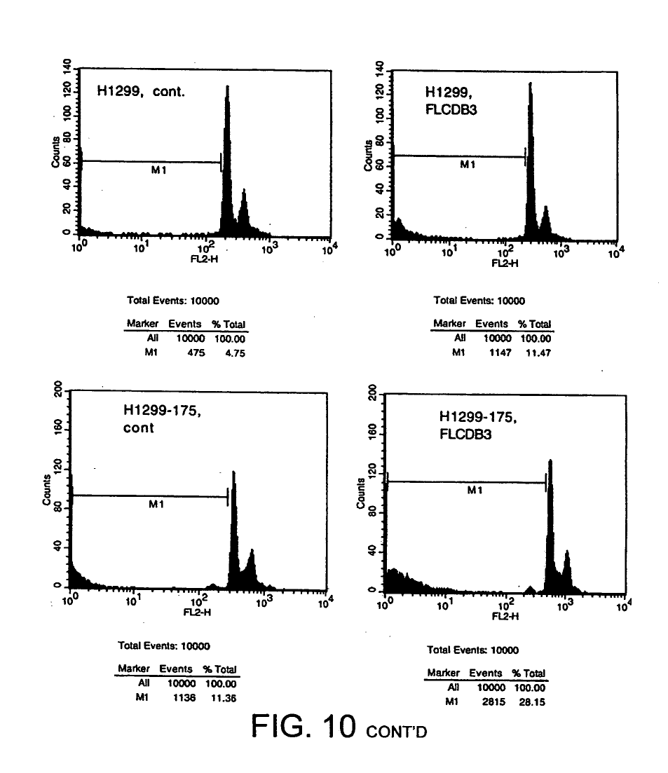

From the results of this experiment, it is clear that CDB3 peptide induces apoptosis in tumour cells in p53-dependent manner (FIG. 10). There is a difference between p53-positive and p53-negative cells. Surprisingly, no growth arrest was detected.

TABLE 1

The peptides tested for binding p53 core domain

| | | |
|---|---|---|
| Peptide | derived from | sequence |

Peptides derived from 53BP2

| | | |
|---|---|---|
| CDB1 | 53BP2 422-428 | MTYSDMQ-NH₂ |
| CDB2 | 53BP2 469-477 | YEPQNDDEL-NH₂ |
| CDB3 | 53BP2 490-498 | REDEDEIEW-NH₂ |

Peptides derived from p53

| | | |
|---|---|---|
| CDB4 | p53 81-100 | TPAAPAPAPSWPLSSSVPSQ-NH₂ |
| CDB5 | phospho-Ser378-p53 369-383 | LKSKKGQSTpSRHKKL-NH₂ |
| CDB6 | phospho-Ser378-p53 361-383 | GSRAHSSHLKSKKGQSTpSR-HKKL-NH₂ |
| CDB7 | p53 369-383 | LKSKKGQSTSRHKKL-NH₂ |
| CDB8 | p53 361-383 | GSRAHSSHLKSKKGQSTSR-HKKL-NH₂ |
| CDB9 | p53 81-94 | TPAAPAPAPSWPLS-NH₂ |
| CDB10 | p53 76-94 | APAAPTPAAPAPAPSWPLS-NH₂ |
| CDB11 | fusion p53 82-94 and 369-383 with a GG linker | PAAPAPAPSWPLSGGLKSKKG-QSTSRHKKL-NH₂ |

TABLE 2
Dissociation constants (K_{d}) for binding of FL-CDB3
to wild-type (WI) and mutant p53¹

| Protein | conditions | K_{d} (μM) |
|---|---|---|
| WT core (94-312) | | 0.53 ± 0.09 |
| WT core + tet (94-363) | | 0.77 ± 0.09 |
| WT core | 4 M urea | 61 ± 10 |
| WT core | 2 M Gdm Cl | >1000 |
| G245S (94-312) | | 0.57 ± 0.09 |
| G245S (94-312) | 4 M urea | 39 ± 4 |
| G245S (94-312) | 2 M Gdm Cl | >1000 |
| R249S (94-312) | | 3.33 ± 0.5 |

¹K_{d} values are determined from the anisotropy and fluorescence at 525 nm following titration of p53 into fluorescein labelled CDB3.

TABLE 3
Dissociation constants (K_{d}) for
CDB3 variants binding to p53 core domain

| Ligand | K_{d} (μM) |
|---|---|
| FL-CDB3¹ | 0.53 ± 0.09 |
| Unlabeled CDB3² | 37 ± 4 |
| biotinylated CDB3³ | 12 ± 1 |
| Immobilized biotinylated CDB3³ | ˜0.2 |
| Fluorescein | >1000 |
| Unlabeled CDB3⁴, 20° C. | 50 ± 9 |

¹Determined by fluorescence anisotropy (see Table 1).
²Determined by anisotropy experiment, in competition with fluorescein labeled CDB3.
³Binding to immobilized peptide as determined from half saturation concentration by BIAcore.
⁴Determined by competition BIAcore at 20° C.

### References

Abarzua, P., LoSardo, J. E., Gubler, M. L., Spathis, R., Lu, Y. A., Felix, A. and Neri, A. (1996) Restoration of the transcription activation function to mutant p53 in human cancer cells. Oncogene, 13, 2477-2482.
Bayle, J. H., Elenbaas, B. and Levine, A. J. (1995) The carboxyl-terminal domain of the p53 protein regulates sequence-specific DNA binding through its nonspecific nucleic acid-binding activity. Proc Natl Acad Sci U S A, 92, 5729-5733.
Bullock, A. N. and Fersht, A. R. (2001) Rescuing the function of mutant p53. Nature Cancer Reviews, 1, 68-76.
Bullock, A. N., Henckel, J., DeDecker, B. S., Johnson, C. M., Nikolova, P. V., Proctor, M. R., Lane, D. P. and Fersht, A. R. (1997) Thermodynamic stability of wild-type and mutant p53 core domain. Proc Natl Acad Sci U S A, 94, 14338-14342.
Bullock, A. N., Henckel, J. and Fersht, A. R. (2000) Quantitative analysis of residual folding and DNA binding in mutant p53 core domain: definition of mutant states for rescue in cancer therapy. Oncogene, 19, 1245-1256.
Cho, Y., Gorina, S., Jeffrey, P. D. and Pavletich, N. P. (1994) Crystal structure of a p53 tumor suppressor-DNA complex: understanding tumorigenic mutations. Science, 265, 346-355.
Foster, B. A., Coffey, H. A., Morin, M. J. and Rastinejad, F. (1999) Pharmacological rescue of mutant p53 conformation and function. Science, 286, 2507-2510.
Gorina, S. and Pavletich, N. P. (1996) Structure of the p53 tumor suppressor bound to the ankyrin and SH3 domains of 53BP2. Science, 274, 1001-1005.
Guex, N. and Peitsch, M. C. (1997) SWISS-MODEL and the Swiss-Pdb Viewer: an environment for comparative protein modeling. Electrophoresis, 18, 2714-2723.
Hainaut, P. and Hollstein, M. (2000) p53 and human cancer: the first ten thousand mutations. Adv Cancer Res, 77, 81-137.
Hupp, T. R., Lane, D. P. and Ball, K. L. (2000) Strategies for manipulating the p53 pathway in the treatment of human cancer. Biochem J, 352 Pt 1, 1-17.
Hupp, T. R., Sparks, A. and Lane, D. P. (1995) Small peptides activate the latent sequence-specific DNA binding function of p53. Cell, 83, 237-245.
Iwabuchi, K., Bartel, P. L., Li, B., Marraccino, R. and Fields, S. (1994) Two cellular proteins that bind to wild-type but not mutant p53. Proc Natl Acad Sci U S A, 91, 6098-6102.
Iwabuchi, K., Li, B., Massa, H. F., Trask, B. J., Date, T. and Fields, S. (1998) Stimulation of p53-mediated transcriptional activation by the p53-binding proteins, 53BP1 and 53BP2. J Biol Chem, 273, 26061-26068.
Kim, A. L., Raffo, A. J., Brandt-Rauf, P. W., Pincus, M. R., Monaco, R., Abarzua, P. and Fine, R. L. (1999) Conformational and molecular basis for induction of apoptosis by a p53 C-terminal peptide in human cancer cells. J Biol Chem, 274, 34924-34931.
Lopez, C. D., Ao, Y., Rohde, L. H., Perez, T. D., O'Connor, D. J., Lu, X;, Ford, J. M. and Naumovski, L. (2000) Proapoptotic p53-interacting protein 53BP2 is induced by UV irradiation but suppressed by p53. Mol Cell Biol, 20, 8018-8025.
Muller-Tiemann, B. F., Halazonetis, T. D. and Elting, J. J. (1998) Identification of an additional negative regulatory region for p53 sequence-specific DNA binding. Proc Natl Acad Sci U S A, 95, 6079-6084.
Nieba, L., Krebber, A. and Pluckthun, A. (1996) Competition BIAcore for measuring true affinities: large differences from values determined from binding kinetics. Anal Biochem, 234, 155-165.
Selivanova, C., Iotsova, V., Okan, I., Fritsche, M., Strom, M., Groner, B., Grafstrom, R. C. and Wiman, K. G. (1997) Restoration of the growth suppression function of mutant p53 by a synthetic peptide derived from the p53 C-terminal domain. Nat Med, 3, 632-638.
Selivanova, C., Ryabchenko, L., Jansson, E., Iotsova, V. and Wiman, K. G. (1999) Reactivation of mutant p53 through interaction of a C-terminal peptide with the core domain. Mol Cell Biol, 19, 3395-3402.
Sigal, A. and Rotter, V. (2000) Oncogenic mutations of the p53 tumor suppressor: the demons of the guardian of the genome. Cancer Res, 60, 6788-6793.
Takenaka, I., Monin, F., Seizinger, B R. and Kley, N. (1995) Regulation of the sequence-specific DNA binding function of p53 by protein kinase C and protein phosphatases. J Biol Chem, 270, 5405-5411.
Wong, K. B., DeDecker, B. S., Freund, S. M., Proctor, M. R., Bycroft, M. and Fersht, A. R. (1999) Hot-spot mutants of p53 core domain evince characteristic local structural changes. Proc Natl Acad Sci U S A, 96, 8438-8442.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and molecular biology or related fields are intended to be within the scope of the following claims.

1. A method of stabilising the native state of a polypeptide, the method comprising exposing the polypeptide to a stabilising molecule that binds to the polypeptide at a site which at least partially overlaps a functional site in the native state of the polypeptide.

2. The method of claim 1, in which the polypeptide is reversibly denatured such that it exists in a native state and a denatured state, in which the stabilising molecule does not bind to the polypeptide in its denatured state.

3. A method of increasing the concentration of a native state of a reversibly denatured polypeptide in a system, in which the system comprises the polypeptide in a first, native state and a second, denatured state, the method comprising:
(a) providing a stabilising molecule which binds to the polypeptide at a site which at least partially overlaps with a functional site in the first native state and thereby stabilising the first, native state of the polypeptide; and
(b) contacting the stabilising molecule with the polypeptide, whereby the concentration of the polypeptide in its native state is incresased.

4. A method of restoring a wild-type phenotype of an organism comprising a mutation in a polypeptide, in which the mutation results in denaturation of the polypeptide and a mutant phenotype, the method comprising exposing the organism or part of the organism to a stabilising molecule which binds to the polypeptide in its native state at a site which at least partially overlaps a functional site of said polypeptide and thereby stabilises the native state of the polypeptide.

5. A method of treatment of a disease in a patient, in which the disease is caused by or associated with a mutation in a polypeptide which results in denaturation of the polypeptide, the method comprising administering to the patient a stabilising molecule which binds to the polypeptide at a site which at least partially overlaps a functional site in its native state and thereby stabilises the native state of the polypeptide.

6. The method of claim 1, in which the stabilising molecule is not a natural binding partner of the polypeptide.

7. The method of claim 1, in which the stabilising molecule consists of a fragment of a natural binding partner of the polypeptide.

8. The method of claim 1, in which the stabilising molecule is a polypeptide engineered to include a polypeptide binding domain of a natural binding partner of the polypeptide.

9. The method of claim 8 wherein said polypeptide binding domain is a binding loop of said natural binding partner of said polypeptide.

10. The method according to claim 1, in which the stabilising molecule is exposed to the polypeptide in presence of a natural binding partner of the polypeptide.

11. The method of claim 1, in which the affinity of binding between stabilising molecule and the polypeptide or site is less than the affinity of a natural binding partner of the polypeptide and the polypeptide or the binding site.

12. The method of claim 1, in which binding between the stabilising molecule and the binding site stabilises the polypeptide and thereby permits binding between the polypeptide and a natural binding partner.

13. The method of claim 1, in which binding between the polypeptide and a natural binding partner stabilises the native state of the polypeptide.

14. A method of assisting the binding between a polypeptide and a natural binding partner for the polypeptide, the method comprising stabilising a native state of the polypeptide by a method according to claim 1, and exposing the stabilised polypeptide to the natural binding partner, wherein binding of the polypeptide to the natural binding partner is assisted.

15. A method of assisting the binding between a polypeptide and a first molecule, in which the polypeptide exists in a native state and a denatured state, the method comprising:
(a) providing a second stabilising molecule that binds to a site which at least partially overlaps a functional site in the native state of the polypeptide;
(b) permitting the second stabilising molecule to bind to the polypeptide to form a complex, thereby stabilising the native state of the polypeptide;
(c) exposing the polypeptide and bound second stabilising molecule complex to the first molecule; and
(d) permitting the first molecule to bind to the polypeptide and thereby displacing the second stabilising molecule, wherein binding between the polypeptide and the first molecule is assisted.

16. The method of claim 1, 14 or 15, in which the functional site comprises or at least partially overlaps with a structural domain, a protein binding domain, a nucleic acid binding domain, or an active site of an enzyme.

17. The method according to claim 15, in which the functional site is essential to the structure or activity, or both, of the polypeptide.

18. The method according to claim 1, 14 or 15, in which the polypeptide comprises an oncogenic protein or a tumour suppressor protein.

19. The method according to claim 18, in which the polypeptide is p53.

20. The method of claim 18, in which the polypeptide is p53 which comprises a mutation, selected from R175H, G245S, R248Q, R249S, R273H, R282W and I1951, which mutation results in reversible denaturation of the polypeptide.

21. The method of any one of claims 1, 14 and 15, in which the stabilising molecule comprises a CDB3 polypeptide having the sequence REDEDEIEW.

22. The method of any one of claims 1, 14 and 15 wherein said stabilising molecule comprises an organic or inorganic small molecule, a natural or derivatised carbohydrate, protein, polypeptide, peptide, glycoprotein, nucleic acid, DNA, RNA, oligonucleotide or protein-nucleic acid (PNA).

23. The method of claim 22 wherein said stabilising molecule is derivatised with a sugar, phosphate, amine, amide, sulphate, sulphide, biotin, a fluorophore or a chromophore.

24. A stabilising molecule which binds to and stabilises the native state of a polypeptide, but not a denatured state of the polypeptide, in which the stabilising molecule binds to a site which at least partially overlaps a functional site of the polypeptide, and in which the stabilising molecule does not consist of a natural binding partner of the polypeptide.

25. The stabilising molecule of claim 24, in which the polypeptide is p53.

26. The stabilising molecule of claim 25, in which the polypeptide is p53 which comprises a mutation, selected from R175H, G245S, R248Q, R249S, R273H, R282W and I195T in which the mutation results in reversible denaturation of the polypeptide.

27. The stabilising molecule of claim 24, in which the stabilising molecule comprises a CDB3 polypeptide having the sequence REDEDEIEW.

28. The stabilising molecule of claim 24, wherein said molecule comprises an organic or inorganic small molecule, a natural or derivatised carbohydrate, protein, polypeptide, peptide, glycoprotein, nucleic acid, DNA, RNA, oligonucleotide or protein-nucleic acid (PNA).

29. The stabilising molecule of claim 28 wherein said molecule is derivatised with a sugar, phosphate, amine, amide, sulphate, sulphide, biotin, a fluorophore or a chromophore.

30. A method of identifying a stabilising molecule that stabilises a polypeptide, the polypeptide is reversibly denaturable such that it exists in a native state and a denatured state, the method comprising the steps of:
(a) providing a native state of the polypeptide comprising a functional site;
(b) exposing the polypeptide to a candidate stabilising molecule;
(c) selecting a candidate stabilising molecule which binds to a site which at least partially overlaps said functional site of the native state of the polypeptide; and
(d) determining whether such binding stabilises the native state of the polypeptide.

31. A method of identifying a stabilising molecule that stabilises a polypeptide, wherein the polypeptide is reversibly denaturable, such that it exists in a native state and a denatured state, the method comprising the steps of:
(a) identifying a functional site of the polypeptide and providing a polypeptide fragment comprising the functional site;
(b) selecting a candidate stabilising molecule which binds to the polypeptide fragment at a site which at least partially overlaps said functional site; and
(c) determining whether the selected candidate stabilising molecule stabilises a native state of the polypeptide.

32. The method of claim 30 or 31 wherein said stabilising molecule comprises an organic or inorganic small molecule, a natural or derivatised carbohydrate, protein, polypeptide, peptide, glycoprotein, nucleic acid, DNA, RNA, oligonucleotide or protein-nucleic acid (PNA).

33. The method of claim 32 wherein said stabilising molecule is derivatised with a sugar, phosphate, amine, amide, sulphate, sulphide, biotin, a fluorophore or a chromophore.

34. The method of claim 31, in which the polypeptide fragment comprising the functional site includes a binding site for a natural binding partner of the polypeptide.

35. A method of claim 22 in which the stabilising molecule is derivatised with a fluorophore.

36. A method of claim 32 in which the stabilising molecule is derivatised with a fluorophore.

37. The methods of claim 35 wherein said stabilising molecule is derivatised with fluorescein.

38. The methods of claim 36 wherein said stabilising molecule is derivatised with fluorescein.

39. The stabilising molecule of claim 24 in which the stabilising molecule is derivatised with a fluorophore.

40. The stabilising molecule of claim 39 wherein said fluorophore is fluorescein.

41. The method of any one of claims 1, 14, 15, 30, or 31, further comprising detecting the binding of a stabilising molecule to the polypeptide using NMR spectroscopy, fluorescence anisotropy, surface plasmon resonance, or Differential Scanning Calorimetry (DSC).

42. A method of treating a disease, the method comprising administering a therapeutic amount of a stabilising molecule of claim 24 to an individual, wherein said disease is treated.

43. The method of claim 5 or 40 wherein the disease is cancer.

44. A pharmaceutical composition comprising a stabilising molecule according to claim 24, together with a pharmaceutically acceptable carrier, diluent or excipient.

45. A pharmaceutical composition comprising a CDB3 polypeptide having the sequence REDEDEIEW together with a pharmaceutically acceptable carrier, diluent or excipient.

46. A method for inducing the onset or progression of apoptosis in one or more cells comprising the step of contacting those one or more cells with a stabilising molecule of claim 24.

47. The method of claim 44 wherein the stabilising molecule is CDB3 peptide.
